(19) United States
(12) Patent Application Publication
(10) Pub. No.: US 2004/0146873 A1
(43) Pub. Date: Jul. 29, 2004

(54) Advanced sleep phase syndrome gen in humans

(76) Inventors: Louis Ptacek, Salt Lake City, UT (US); Ying-Hui Fu, Salt Lake City, UT (US); Christopher Jones, Salt Lake City, UT (US); David Virshup, Salt Lake City, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/451,689

(22) PCT Filed: Jan. 11, 2002
(86) PCT No.: PCT/US02/00741

(51) Int. Cl. C12Q 1/68

(52) U.S. Cl. 435/6

(57) ABSTRACT

The present invention includes the disclosure of the hPER2 gene and a mutant of the hPER2 gene that participates in the human circadian biological clock. The product of the mutant hPER2 gene found in some familial advanced sleep phase syndrome patients is hypophosphorylated by casein kinase epsilon due to the serine-to-glycine mutation caused by the point mutation of the genomic sequence. Specifically, this serine-to-glycine mutation affects the casein kinase epsilon binding region of the hPER2 protein, thus blocking the phosphorylation cascade ordinarily caused by the binding of casein kinase epsilon to hPER2.

### RELATED APPLICATIONS

This application claims the benefit of U.S. Provisional Application Serial No. 60/261,054, filed Jan. 11, 2001, and entitled “Identification of an Advanced Sleep Phase Syndrome Gene in Humans,” which is incorporated herein by reference.

### FIELD OF THE INVENTION

The present invention relates to a gene involved in the human circadian biological clock. Specifically, the present invention includes the hPER2 gene and a mutant of the hPER2 gene that participates in the human circadian biological clock.

### TECHNICAL BACKGROUND

The International Classification of Sleep Disorders lists approximately 60 disorders of human sleep. Association, A.S.D., International classification of sleep disorders: Diagnostic and coding manual, 1997, Rochester. The main categories of sleep-wake complaint in clinical practice are excessive daytime sleepiness (EDS), difficulty initiating and/or maintaining sleep (DIMS), and unwanted behaviors arising out of sleep. The most common of these sleep disorders are obstructive sleep apnea (with EDS), anxious and depressive features (with DIMS), restless legs syndrome (with DIMS and/or EDS), narcolepsy (with EDS), and the circadian (i.e. daily sleep schedule) disorders of either delayed or advanced sleep phase syndromes (DSPS or ASPS). Circadian sleep schedule disorders are common in young and elderly patients alike, and often cause significant sleep deprivation. The behavioral, cognitive and memory impairments caused by sleep deprivation have been shown to adversely affect driving and work safety, social function, school performance, and overall quality of life.

The master circadian pacemaker in mammals is located in the suprachiasmatic nucleus (SCN) of the anterior hypothalamus. Ibuka & Kawamura, Loss of circadian rhythm in sleep-wakefulness cycle in the rat by suprachiasmatic nucleus lesions, Brain Res., 1975, 96(1):76-81. The SCN rhythms of firing rate and gene expression, and thus the sleep-wake and other bodily rhythms, are entrained to the 24-hour solar day primarily via photic information. This information is most likely transduced by unknown retinal ambient light receptors. Czeisler, C. A., et al., Bright light induction of strong (type 0) resetting of the human circadian pacemaker, Science, 1989, 244(4910):1328-33; Moore, R. Y., Retinohypothalamic projection in mammals: a comparative study, Brain Res., 1973, 49(2):403-9.

The circadian rhythm of alertness normally includes a seemingly paradoxical nadir of sleepiness at the end of the day, called the “Maintenance of Wakefulness Zone.” Edgar, D. M. et al., Effect of SCN lesions on sleep in squirrel monkeys: evidence for opponent processes in sleep-wake regulation, J. Neuroscience, 1993, 13:1065-1079. Following this, there is a peak in difficulty sustaining wakefulness in the second third of the sleep period, from approximately 3:00-5:00 a.m. and then a gradual increase in alertness until the next evening. Pineal release of melatonin is known to be stimulated by the SCN starting at about 1-2 hours before habitual sleep onset time and continuing through the night unless such stimulation is masked by light of more than approximately 50-100 lux intensity. Lewy, A. J., et al., Light suppresses melatonin secretion in humans, Science, 1980, 210(4475):1267-9. Thus, the increase in melatonin blood levels in dim light (DLMO) is thought to be a marker of biological circadian (SCN) time as opposed to the actual time of the 24-hour solar “day.”

The observation has been that, in most cases of advanced phase sleep syndrome, or “ASPS,” and delayed sleep phase syndrome, or “DSPS,” the entire sleep-wake cycle is shifted either earlier or later, respectively, with respect to solar time. The phenomenon of “internal desynchronization” of the sleep-wake rhythm from the melatonin or temperature rhythms has led to the notion that the former is less tightly coupled to the SCN rhythm than the latter, thus making it necessary to measure the phase of both the sleep-wake and the melatonin or temperature rhythms to more fully describe how the circadian system is functioning. Wever, R. A., The circadian system in man, results of experiments under temporal isolation, 1979, Heidelberg: Springer-Verlag.

Individuals affected by the sleep phase disorders noted above are characterized by several traits. DSPS patients feel wide awake, energetic and motivated until late in the night. As a result and depending on the severity, sleep onset may be delayed until 1:00 to 6:00 a.m., and the circadian “morning” increase in alertness does not occur until approximately 10:00 a.m. to 2:00 p.m. Sleep phase-delayed individuals are often sleep deprived because sleep onset is delayed by the biological clock and morning wake up time is enforced by the alarm clock and social responsibilities. The prevalence of DSPS in the general population is thought to be high, especially in adolescents and young adults, but the precise prevalence is not known. There is currently much discussion in school districts across the country about whether school start times should be delayed for adolescents in order to increase their nightly sleep time and thus their academic and social performance. Foundation, N. S., Adolescent sleep needs and patterns, 2000, National Sleep Foundation: Washington, D.C.

People with ASPS fall asleep during what would be the “Maintenance of Wakefulness Zone” for conventional sleepers and tend to wake up alert and energetic in the early morning hours when most people are the sleepiest. ASPS patients are often presented with the difficulties both of staying awake to satisfy domestic responsibilities in the evening and of an obligate early morning awakening before other people are active. This can result in significant sleep deprivation if social responsibilities keep the patient awake late and their biological clock wakes them up early. Some people with ASPS sleep on their “biological” schedule, do not complain, and find that they can accomplish a great deal in the early morning without other people interrupting them. Therefore, ASPS may be seen as a condition or trait, and not always as a disabling “disorder.”

The most common cause of ASPS is the natural aging process, which is also associated with phase advance of the temperature rhythm. Czeisler, C. A., et al., Association of sleep-wake habits in older people with changes in output of circadian pacemaker, Lancet, 1992, 340(8825):933-6. The prevalence of ASPS in the elderly is high, but the precise prevalence is not known. The pathophysiology is also unknown, but recent evidence suggests that a shorter endogenous circadian period length, tau (τ), i.e. a “faster clock”is not the explanation. Czeisler, C. A., et al., Stability, precision, and near-24-hour period of the human circadian pacemaker, Science, 1999, 284(5423):2177-81.

The spectrum of sleep schedule preference in the normal and younger population also includes many people with a modest “morning lark” tendency. Weak polygenic influences are suspected to be a cause of this characteristic based in part on heredibility studies in twins and on candidate gene polymorphism correlations in large populations of apparently normal sleepers. Selby, J., et al., Morningness/eveningness is heritable, Society for Neuroscience Abstracts, 1992, 18:196; Katzenberg, D., et al., A CLOCK polymorphism associated with human diurnal preference, Sleep, 1998, 21(6):569-76. Autosomal dominant ASPS with profound sleep phase advance has been documented, but appears to be uncommon. Jones, C. R., et al., Familial advanced sleep-phase syndrome. A short-period circadian rhythm variant in humans, Nat. Med., 1999, 5(9):1062-5. In one subject, a remarkably short τ was the apparent explanation for the phase advance. There are only isolated case reports of post-traumatic ASPS. Govindan, S. and E. Govindan, Brain imaging in post traumatic circadian rhythm sleep disorders, Sleep Research, 1995, 24:A308.

Circadian dysrhythmias other than DSPS and ASPS include the non-24 hour sleep-wake disorder and imposed perturbations such as shift work schedules and “jet lag”. A non-24 hour sleep-wake schedule is seen in approximately 50% of people with complete retinal blindness. Sack, R. L., et al., Entrainment of free-running circadian rhythms by melatonin in blind people, N. Engl. J. Med., 2000, 343(15):1070-7. By some estimates, up to 20% of the work force is on some form of shift work schedule. Mellor, E. F., Shift work and flexitime: how prevalent are they?, in Monthly Labor Review, 1986, pp. 14-21. Additionally, transmeridian flight is popular among the traveling public.

The correlations between genotype and different aspects of circadian phenotype in different genetic causes of familial advanced sleep phase syndrome, or “FASPS” that would help elucidate molecular circadian mechanisms are either unknown or poorly described. For example, the variability in phase advance within one kindred with highly penetrant monogenic FASPS was shown by the inventors to be considerable, thus suggesting that polymorphisms in other candidate genes and/or environmental factors also influence the magnitude of phase advance. Jones, C. R., et al., Familial advanced sleep-phase syndrome. A short-period circadian rhythm variant in humans, Nat. Med., 1999.5(9): 1062-5. Whether such variability will be seen in other FASPS kindreds is unknown. It would also be of interest to compare the average severity of phase advance produced by different human ASPS mutations since currently only one mutation is known. Preliminary data gathered by the inventors demonstrated significantly more phase advance after just one day of imposed early evening dim light in FASPS subjects than controls. Id. Differences in this tendency for rapid phase advance could shed light on the how the formal properties of the clock are affected by different mutations. Three subtypes of ASPS based on differences in the phase angle of entrainment of the sleep-wake rhythm relative to the melatonin rhythm were predicted 10 years ago. Limited abstract and unpublished data lend support to two of these subtypes. Id., Lewy, A. J., Chronobiologic disorders, social cues, and the light-dark cycle, Chronobiol. Int., 1990, 7(1):15-21; Lewy, A. J., et al., Later circadian phase of plasma melatonin relative to usual waketime in older subjects, Sleep, 2000, 23:A188 (data not shown).

Additional descriptions of qualitatively different sleep vs. melatonin phase relationships in different ASPS mutations could therefore add new subtypes to the nosology of circadian dysrhythmias. Computer simulations and limited empirical human data support a relationship between a shorter endogenous τ and an earlier phase angle of entrainment of the sleep-wake and melatonin rhythms relative to the light-dark cycle. Klerman, E. B., et al., Simulations of light effects on the human circadian pacemaker: implications for assessment of intrinsic period, Am. J. Physiol., 1996, 270(1 Pt 2):R271-82; Sack, R. L., R. W. Brandes, and A. J. Lewy, Correlation of intrinsic circadian period with morningness-eveningness in young men, Sleep, 1999, 22:S92; Duffy, J., et al., Correlation of intrinsic circadian period with morningness-eveningness in young men, Sleep, 1999, 22(Suppl 1):S92.

Describing this relationship in people has been hampered by the relatively small range of τ among normal volunteers. The availability of human FASPS mutants would help overcome this limitation. It is currently not known how different human FASPS mutations might interact with the common trend toward phase delay during adolescence and phase advance during the geriatric years. A description of whether some FASPS mutations seem to be clinically silent during adolescence, or have striking progression of phase advance with age beyond the fifth decade might generate hypotheses on the molecular mechanisms of these common ontogenetic/age-related changes in circadian organization.

Several model systems of human circadian sleep disorders have been developed in various organisms. Specifically, mutagenesis screens have led to the molecular characterization of essential clock genes in Drosophila melanogaster, Neurospora crassa, Chlamydomonas, Cyanobacteria, and Arabidopsis. Bruce, V. G., Mutants of the biological clock in Chlamydomonas reinhardi, Genetics, 1972, 70:537-548; Kondo, T., et al., Circadian clock mutants of cyanobacteria, Science, 1994, 266(5188):1233-6; and Millar, A. J., et al., Circadian clock mutants in Arabidopsis identified by luciferase imaging, Science, 1995, 267(5201): 1161-3. For Drosophila, two genes that are central to the circadian clock, period (per) and timeless (tim), have been identified as the result of ethyl methane sulfonate and transposable P-element mutagenesis screens, respectively. Sehgal, A., et al., Loss of circadian behavioral rhythms and per RNA oscillations in the Drosophila mutant timeless, Science, 1994, 263(5153):1603-6; Konopka, R. J. and S. Benzer, Clock mutants of Drosophila melanogaster, Proc. Natl. Acad. Sci. USA, 1971, 68:2112-2116.

Similarly, the frequency (frq) gene was identified following a nitrosoguanidine mutagenesis screen in Neurospora. Feldman, J. F. and M. N. Hoyle, Isolation of circadian clock mutants of Neurospora crassa, Genetics, 1973, 75:605-613. All three of these genes were subsequently cloned and the mutations causing the aberrant circadian phenotypes have been identified. Baylies, M. K., et al., Changes in abundance or structure of the per gene product can alter periodicity of the Drosophila clock, Nature, 1987, 326(6111):390-2; McClung, C. R., B. A. Fox, and J. C. Dunlap, The Neurospora clock gene frequency shares a sequence element with the Drosophila clock gene period, Nature, 1989, 339(6225):558-62; Myers, M. P., et al., Positional cloning and sequence analysis of the Drosophila clock gene, timeless, Science, 1995, 270(5237):805-8; and Yu, Q., et al., Molecular mapping of point mutations in the period gene that stop or speed up biological clocks in Drosophila melanogaster, Proc. Natl. Acad. Sci. U.S.A., 1987, 84(3):784-8. It is of note that for both per and frq, different alleles can result in either short or long endogenous period.

The development of the murine clock is genetically programmed independently of the environment, and studies of inbred mouse strains indicate that one or more genetic loci influence τ. Davis, R. C. and M. Menaker, Development of the mouse circadian pacemaker: independence from environmental cycles, J. Comp. Physiol., 1981, 143:527-539. Takahashi and colleagues initiated an N-ethyl-N-nitrosourea (ENU) mutagenesis screening strategy to isolate clock mutations in the mouse. Takahashi, J. S., L. H. Pinto, and M. H. Vitaterna, Forward and reverse genetic approaches to behavior in the mouse, Science, 1994, 264(5166):1724-33. They were successful in identifying a mutation, designated clock (Clk) that has several effects on the circadian behavior of mice. Vitaterna, M. H., et al., Mutagenesis and mapping of a mouse gene, Clock, essential for circadian behavior, Science, 1994, 264(5159):719-25.

Clk is a semidominant mutation and lengthens the period of the circadian rhythm by about one hour in clk/+ heterozygotes. In clk/clk homozygotes, the period lengthens by about four hours upon initial transfer to constant darkness, after which these mice lose persistent circadian rhythms. The mutant allele thus competes with the wild-type allele of the gene in the generation of circadian rhythms, providing strong evidence that the clk gene is an essential component of the mammalian circadian clock system. Id. Using a similar strategy, another laboratory generated a mutant mouse (Wheels) with a lengthened τ, which, unlike clk, exhibits a complex phenotype including bidirectional circling, hyperactivity, and inner ear abnormalities. Nolan, P. M., et al., Heterozygosity mapping of partially congenic lines: mapping of a semidominant neurological mutation, Wheels (Whl), on mouse chromosome 4, Genetics, 1995, 140(1):245-54.

A vertebrate single-gene mutation that shortened τ was discovered as a spontaneous, autosomal, semidominant allele in golden hamsters (tau). In this model, the τ of temperature and locomotor rhythmicity is shortened to about 22 hours in heterozygotes and 20 hours in homozygotes. Ralph, M. R. and M. Menaker, A mutation of the circadian system in golden hamsters, Science, 1988, 241(4870):1225-7. These animals cannot entrain to the 24-hour light-dark cycle, and photoperiodic responsiveness is also dramatically altered. Menaker, M. and R. Refinetti, The tau mutation in golden hamsters, Molecular genetics of biological rhythms, ed. M. W. Young, 1993, 255-269. Interpulse intervals in the secretion of luteinizing hormone and cortisol are lengthened, while other rhythmic phenomena (estrous cyclicity, heart rate) remain unaffected. Loudon, A. S., et al., Ultradian endocrine rhythms are altered by a circadian mutation in the Syrian hamster, Endocrinology, 1994, 135(2):712-8; Refinetti, R. and M. Menaker, Evidence for separate control of estrous and circadian periodicity in the golden hamster, Behav. Neural Biol., 1992, 58(1):27-36; and Refinetti, R. and M. Menaker, Independence of heart rate and circadian period in the golden hamster, Am. J. Physiol., 1993, 264(2 Pt 2):R235-8.

A shortened τ is one possible cause of ASPS. Stable entrainment to the light-dark cycle when τ is short is only possible when the circadian cycle, including sleep, is advanced because this exposes more of the phase-delay portion of the phase-response curve to remaining afternoon and evening light. Thus the short period τ mutant hamster is an excellent model of ASPS in humans. However, for the circadian rhythm genes per and frq, both long and short alleles are recognized. Therefore, homologues of the clk and wheels genes (and other circadian rhythm gene homologues) are still excellent candidates for ASPS.

Within the last few years, there has been an explosion of new data regarding clock genes and mechanisms in a variety of organisms. Reppert, S. M., A clockwork explosion! Neuron, 1998, 21(1):1-4, Wager-Smith, K. and S. A. Kay, Circadian rhythm genetics: from flies to mice to humans, Nat. Genet., 2000, 26(1):23-7. Several proteins have been identified to be central to the design of the clock. In Drosophila, these include PER and TIM which act to repress transcription of their own genes in a negative feedback loop (FIG. 1A). They intermittently engage and disengage from transcriptional activators (CLK, CYC or BMAL) to form a dynamic multiprotein complex. Lee, C., K. Bae, and I. Edery, PER and TIM inhibit the DNA binding activity of a Drosophila CLOCK-CYC/dBMAL1 heterodimer without disrupting formation of the heterodimer: a basis for circadian transcription, Mol. Cell Biol., 1999, 19(8):5316-25. The “lag” produced between the transcriptional induction of per and tim and the nuclear translocation of the repressor proteins they encode creates a temporal separation between phases of induction and repression. This temporal separation therefore generates the important feature in the clock mechanism: oscillation. Dunlap, J. C., Molecular bases for circadian clocks, Cell, 1999, 96(2):271-90.

Among all species that have been studied, the Drosophila clock is best understood. Scully, A. L. and S. A. Kay, Time flies for Drosophila, Cell, 2000, 100(3):297-300. At around noon, the CLK protein together with its partner, CYC, bind to E-box DNA elements and activate a slow transcriptional induction of the per and tim genes. Lee, C., K. Bae, and I. Edery, PER and TIM inhibit the DNA binding activity of a Drosophila CLOCK-CYC/dBMAL1 heterodimer without disrupting formation of the heterodimer: a basis for circadian transcription, Mol. Cell Biol., 1999, 19(8):5316-25; Hao, H., D. L. Allen, and P. E. Hardin, A circadian enhancer mediates PER-dependent mRNA cycling in Drosophila melanogaster, Mol. Cell Biol., 1997, 17(7):3687-93, and Rutila, J. E., et al., CYCLE is a second bHLH-PAS clock protein essential for circadian rhythmicity and transcription of Drosophila period and timeless, Cell, 1998, 93(5):805-14. Per and tim RNA levels begin to rise, but DBT (a constitutively produced protein homologous to casein kinase 1ε reduces the stability (and thus the level of accumulation) of monomeric PER protein by phosphorylation. Price, J. L., et al., double-time is a novel Drosophila clock gene that regulates Period protein accumulation, Cell, 1998, 94(1):83-95. Nightfall allows TIM, a light sensitive protein, to rise to a level at which it can bind and protect PER protein from degradation and stable TIM:PER heterodimers begin to form. Id., Kloss, B., et al., The Drosophila clock gene double-time encodes a protein closely related to human casein kinase I epsilon, Cell, 1998, 94(1):97-107.

By midnight, TIM:PER heterodimers have translocated into the nucleus and have physically associated with CLK:CYC complexes. Young, M. W., The molecular control of circadian behavioral rhythms and their entrainment in Drosophila, Annu. Rev. Biochem., 1998, 67:135-52. This association inhibits the ability of the CLK:CYC protein complex to bind DNA and therefore transcription of these genes ceases. Darlington, T. K., et al., Closing the circadian loop: CLOCK-induced transcription of its own inhibitors per and tim, Science, 1998, 280(5369):1599-603; and Lee, C., K. Bae, and I. Edery, The Drosophila CLOCK protein undergoes daily rhythms in abundance, phosphorylation, and interactions with the PER-TIM complex, Neuron, 1998, 21(4):857-67. The mRNA levels of per and tim then decline throughout the night. Daybreak stimulates the photoreceptor, CRY, and rhodopsin to sequester TIM protein and diminish its function as a transcriptional regulator. Emery, P., et al., CRY, a Drosophila clock and light-regulated cryptochrome, is a major contributor to circadian rhythm resetting and photosensitivity, Cell, 1998, 95(5):669-79; Stanewsky, R., et al., The cryb mutation identifies cryptochrome as a circadian photoreceptor in Drosophila, Cell, 1998, 95(5):681-92. TIM becomes phosphorylated, ubiquitinated and degraded via the proteasomal pathway by the induction of light. Naidoo, N., et al., A role for the proteasome in the light response of the timeless clock protein, Science, 1999, 285(5434): 1737-41. By noon the second day, the levels of PER and TIM have decreased to where they can no longer inhibit CLK:CYC transcription activity and a new cycle of synthesis begins. This self-sustaining loop can be reset by the major entraining cue: light, which causes rapid TIM protein degradation.

The transcriptional regulation of the Drosophila clk gene is the mirror image of that of the per and tim genes. Glossop, N. R., L. C. Lyons, and P. E. Hardin, Interlocked feedback loops within the Drosophila circadian oscillator, Science, 1999, 286(5440):766-8. CLK:CYC repress clk expression, either directly or indirectly. PER and TIM block this repression. Lack of both PER-TIM de-repression and CLK-CYC repression results in high levels of clk mRNA, which implies that a separate clk activator is present. Therefore, the Drosophila circadian feedback loop is composed of two interlocked negative feedback loops. Interestingly, expression of cry cycles in phase with clk, and cry and clk mRNA levels are affected the same way in various clock mutants. Id.; Emery, P., et al., CRY, a Drosophila clock and light-regulated cryptochrome, is a major contributor to circadian rhythm resetting and photosensitivity, Cell, 1998, 95(5):669-79. A newly identified gene (vrille) oscillates in phase with per and tim and has an E box sequence as well. Blau, J. and M. W. Young, Cycling vrille expression is required for a functional Drosophila clock, Cell, 1999, 99(6):661-71. However, the exact role of vrille in the Drosophila clock is not yet understood.

Mammalian clock organization shares some similarities and differences with that of the fly (FIG. 1B). Homologues of the Drosophila circadian clock genes have been identified in mammals including: Clk, Ck1ε (homolog of dbt), Cry1, Cry2, Per1, Per2, Per3, Bmal (homologous to cyc), and Tim (although there is a suggestion that mTim is not the true mammalian homologue of dtim. Shearman, L. P., et al., Interacting molecular loops in the mammalian circadian clock, Science, 2000, 288(5468):1013-9. As in the fly, mammalian CLOCK and BMAL act as transcriptional activators on E-boxes found in mPer and other circadianly regulated promoters. PER negatively regulates the transcriptional activity of CLK and BMAL as in the fly. Dunlap, J. C., Molecular bases for circadian clocks, Cell, 1999, 96(2):271-90.

DBT (homologous to CK1ε) phosphorylates and destabilizes PER in mammals as in flies. Keesler, G. A., et al., Phosphorylation and destabilization of human period I clock protein by human casein kinase I epsilon, Neuroreport, 2000, 11(5):951-5. However, several clock genes that are unique in the fly have multiple homologous copies in the mammalian genome. King, D. P. and J. S. Takahashi, Molecular genetics of circadian rhythms in mammals, Annu. Rev. Neurosci., 2000, 23:713-42. The physical interactions of some of the pacemaker proteins in fly have been found in mouse, but other interactions are specific to one or the other species.

Although the Drosophila and mouse circadian feedback loops have similar components, they function at opposite phases of the circadian cycle and mediate light-dependent phase resetting through different mechanisms. In Drosophila, transcription of the per and tim genes is activated by CLK:CYC late in the day and inhibited by PER and TIM late at night. In contrast, transcription of mPer1, 2, and 3 is activated by CLK:BMAL early in the day and repressed by PER and perhaps TIM late in the day. Dunlap, J. C., Molecular bases for circadian clocks, Cell, 1999, 96(2):271-90. Despite these phase differences, Drosophila and mice show similar responses to light pulses administered during the dark phase. The mechanisms by which light resets the clock are very different in fly and mouse. In Drosophila, light leads to the degradation of TIM protein. dCRY acts as a circadian photoreceptor, resetting the clock through light-dependent interactions with TIM. Hunter Ensor, M., A. Ousley, and A. Sehgal, Regulation of the Drosophila protein timeless suggests a mechanism for resetting the circadian clock by light, Cell, 1996, 84(5):677-85; Lee, C., et al., Resetting the Drosophila clock by photic regulation of PER and a PER-TIM complex, Science, 1996, 271(5256):1740-4; Myers, M. P., et al., Light-induced degradation of TIMELESS and entrainment of the Drosophila circadian clock, Science, 1996, 271(5256): 1736-40; and Zeng, H., et al., A light-entrainment mechanism for the Drosophila circadian clock, Nature, 1996, 380(6570):129-35.

In mice, light causes the rapid induction of mPer1 and mPer2 transcription. Albrecht, U., et al., A differential response of two putative mammalian circadian regulators, mper1 and mper2, to light, Cell, 1997 91(7):1055-64, Shearman, L. P., et al., Two period homologs: circadian expression and photic regulation in the suprachiasmatic nuclei, Neuron, 1997, 19(6):1261-9; Shigeyoshi, Y., et al., Light-induced resetting of a mammalian circadian clock is associated with rapid induction of the mPer1 transcript, Cell, 1997, 91(7):1043-53; and Zylka, M. J., et al., Three period homologs in mammals: differential light responses in the suprachiasmatic circadian clock and oscillating transcripts outside of brain, Neuron, 1998, 20(6): 1103-10. Moreover, recent results show that the Cry genes in mice are required for circadian clock function, but they do not preclude a possible role for these genes in circadian photoreception as well. Okamura, H., et al., Photic induction of mPer1 and mPer2 in cry-deficient mice lacking a biological clock, Science, 1999, 286(5449):2531-4.

In vitro studies revealed that mCRY1 and mCRY2 play two critical roles within the circadian feedback loop itself. First, both mCRY1 and mCRY2 promote translocation of mPER1, 2, and 3 into the nucleus. Second, once in the nucleus, mCRY effectively inhibits transcription (by CLK:BMAL) of reporter genes coupled to the mPer1 promoter. Kume, K., et al., mCRY1 and mCRY2 are essential components of the negative limb of the circadian clock feedback loop, Cell, 1999, 98(2): 193-205. Mouse CRY physically interacts with PER protein and represses transcription of the gene per, as does Drosophila TIM. Hardin, P. E. and N. R. Glossop, Perspectives: neurobiology. The CRYs of flies and mice, Science, 1999, 286(5449):2460-1. The identity of the mammalian circadian photoreceptor(s) is still unclear.

A deletion mutation in the PAS domain of the mouse Per2 gene has been made. Zheng, B., et al., The mPer2 gene encodes a functional component of the mammalian circadian clock, Nature, 1999, 400(6740): 169-73. Mice homozygous for this mutation display a short circadian period and then become arrhythmic in constant darkness. The loss of circadian rhythmicity is not due to a decrease in wheel-running activity and can be reversed by a light pulse. Histological analysis of SCN showed no gross anatomic differences suggesting that the abnormal circadian phenotype is not due to a developmental defect. Rhythmic expression of mPer1 and mPer2 RNA levels was still present but at a very low amplitude consistent with a positive regulatory function of mPER2 in the circadian mechanism. Subsequently, Shearman et al., demonstrated that mPER2 is a positive regulator of a Bmal1 feedback loop that interacts with the CRY and PER feedback loop described above. Shearman, L. P., et al., Interacting molecular loops in the mammalian circadian clock, Science, 2000, 288(5468):1013-9. This level of complexity again underscores the difficulty of predicting the direction and magnitude of change in τ with mutations in candidate genes.

The rhythmic machinery of the clock has been shown to directly regulate the transcription of certain output genes, including two related transcription factors (albumin D-element binding protein in mammals and vrille in flies). Blau, J. and M. W. Young, Cycling vrille expression is required for a functional Drosophila clock, Cell, 1999, 99(6):661-71. The neuropeptide, ‘pigment dispersing factor,’ is circadianly regulated at the level of protein abundance in flies. Park, J. H., et al., Differential regulation of circadian pacemaker output by separate clock genes in Drosophila, Proc. Natl. Acad. Sci. U.S.A., 2000, 97(7):3608-13. Mutations in the genes encoding these three proteins have been found to perturb locomotor rhythmicity. Blau, J. and M. W. Young, Cycling vrille expression is required for a functional Drosophila clock, Cell, 1999, 99(6):661-71; Renn, S. C., et al., A pdf neuropeptide gene mutation and ablation of PDF neurons each cause severe abnormalities of behavioral circadian rhythms in Drosophila [published erratum appears in Cell 2000 Mar 31;101(1):i following 113], Cell, 1999, 99(7):791-802; Franken, P., et al., The transcription factor DBP affects circadian sleep consolidation and rhythmic EEG activity, J. Neurosci., 2000, 20(2):617-25; Lopez-Molina, L., et al., The DBP gene is expressed according to a circadian rhythm in the suprachiasmatic nucleus and influences circadian behavior, Embo. J., 1997, 16(22):6762-71. Another mutation alters the RNA-binding protein, LARK, and affects the circadian rhythm of eclosion of adult flies from their pupal cases, but not the rhythm of locomotion. Newby, L. M. and F. R. Jackson, A new biological rhythm mutant of Drosophila melanogaster that identifies a gene with an essential embryonic function, Genetics, 1993, 135(4):1077-90. The series of steps linking the clock to behavioral rhythms via regulation of clock-controlled genes remains largely unclear in any species.

Familial ASPS represents the first description of a monogenic circadian rhythm disorder in humans. Since ASPS is clearly a single gene Mendelian trait in the families studied, a very focused and directed effort at molecular characterization has an extremely high likelihood of success. Thus, it will provide a window into better understanding of human sleep physiology. The ubiquitous prevalence of circadian rhythms in nature—from primitive unicellular flagellates to insects, mammals, and primates—as well as the adaptive significance of circadian rhythms, suggests the possibility that clock mechanisms are evolutionarily conserved. This hypothesis can be tested once more is known about the molecular basis of human circadian physiology and will complement understanding of circadian rhythms studied in other animals. Molecular characterization of ASPS will lead to better understanding of normal sleep physiology and possibly to a better understanding of other human circadian sleep disorders including geriatric sleep phase advance, the sleep phase delay of adolescence and young adulthood, free-running rhythms of the blind, seasonal affective disease, and other forms of insomnia. This work may also have implications for sleep alterations in these settings.

In summary, despite the fact that the field of circadian rhythm genetics and biology has grown tremendously over the last decade, much of human circadian rhythm genetics is not well understood. Extensive additional study of human circadian rhythm mutations is required to further understand the similarities and difference between human clocks and those of other organisms. It would thus be an advancement in the art to disclose a human gene which participates in circadian rhythm cycles in humans. Such a gene and methods for its use are disclosed herein.

### SUMMARY OF THE DRAWINGS

A more particular description of the invention briefly described above will be rendered by reference to the appended figures. These figures only provide information concerning typical embodiments of the invention and are not therefore to be considered limiting of its scope.

FIG. 1. (A) The Drosophila Clock: Monomeric PER (P) and TIM (T) rhythmically accumulate in the cytoplasm. Phosphorylation of PER by DBT (casein kinase 1ε) alters stability and rates of accumulation of PER. When levels become sufficient, PER and TIM heterodimerize and are then able to translocate into the nucleus where they repress the expression of their own genes. Dimers of CLK and CYC repress transcription of dCLK and drive expression of PER and TIM. The PER:TIM dimers de-repress this regulatory step. Depending on the phase of the circadian cycle, light-dependent (and CRY-dependent) degradation of TIM phase shifts the entire cycle by either delaying the accumulation of PER:TIM dimers or hastening a decrease in their levels. (B) The Mammalian Clock shares similarities with the Drosophila clock. However, there are three mammalian PER homologues compared to the one in Drosophila. In the mammalian clock, PER dimerizes with CRY and apparently not with the protein encoded by the cloned mammalian Tim gene. Casein kinase Iε (similar to the Drosophila dbt) phosphorylates PER proteins.

FIG. 2. ASPS Kindred 2174 is a large Utah pedigree and was the first recognized familial ASPS pedigree. Circles represent women and squares represent men. Blackened circles and squares represent individuals affected with FASPS and empty circles and squares represent unaffected individuals. Unknown individuals (not meeting strict criteria for being ‘affected’ or ‘unaffected’) are eliminated from this pedigree for the sake of simplicity. Horne Östberg scores are shown below individuals. The dotted line marks a branch where a ‘marry-in’ has a striking morning lark phenotype.

FIG. 3. Additional ASPS Kindreds: Circles represent females and squares represent males. The inset key describes the affection status of individuals. An additional 19 probands have been identified, many who have family histories. The size of these families is not yet clear as they are still under active investigation. These reagents along with additional families that were recruited should provide sufficient reagents to identify additional circadian rhythm mutations in ASPS families.

FIG. 4. The genomic structure of hPer2: The entire hPer2 gene has been cloned, and its intron/exon boundaries have been characterized. The gene contains 23 exons, shown in rectangles. The intervening introns are not drawn to scale. The start codon (ATG) is marked in exon 2. The stop codon (TAA) is shown in exon 23. Underneath the gene structure are marks showing the portion of the gene encoding the PAS domain and the CK1ε binding site. The mutation in kindred 2174 (S662G) is marked in exon 17. A delta sign marking exon 22 shows the location of the sequence error (a one base pair deletion) in the published hPer2 cDNA sequence. An additional base pair change was noted in intron 13 (as marked) in one family with ASPS. This base pair change was not noted in a large set of control DNAs (see preliminary results section).

FIG. 5. Amino acid sequences of PER homologues in the region of the S662G mutation are shown. The first sequence for hPER1 is included in the sequence listing as SEQ ID NO: 5, and its analog in mus musculus is included as SEQ ID NO: 6. The sequence for HPER is included in the sequence listing as SEQ ID NO: 7, and its analog in mus musculus is included as SEQ ID NO: 8. The sequence for hPER3 is included in the sequence listing as SEQ ID NO: 9, and its analog in mus musculus is included as SEQ ID NO: 10. In the mutant hPER2 discussed herein, (amino acid sequence in SEQ ID NO: 1, and nucleic acid sequence in SEQ ID NO: 2) the serine at position 662 is replaced by a glycine (G). The wild type hPER2 sequence instead has a serine at position 662. The amino acid sequence of wild type hPER2 is found in SEQ ID NO: 3, and the nucleic acid sequence is found in SEQ ID NO: 4. Four asterisks mark four subsequent serine residues each with two intervening amino acids. The SXXS motif is a binding site for casein kinase 1. Data from study of additional mutants suggests the possibility that some of these subsequent serines may be phosphorylated after phosphorylation of serine at position 662.

FIG. 6. Sequence of hPer2 mutation in kindred 2174. DNA sequences shown from the hPer2 gene. An arrow marks a double peak at position 2106 in the hPer2 cDNA. This A to G transversion predicts substitution of a highly conserved serine residue at amino acid position 662 by a glycine. This double peak was noted when sequencing in both directions. It was seen in all affected individuals in the pedigree and not in any unaffected individuals in the pedigree. SSCP analysis demonstrates the aberrant band arising from this base pair change was not present in the 92 normal controls.

FIG. 7. In vitro CKIε phosphorylation of wild-type and mutant hPER2. In vitro transcribed and translated hPER2 (WT), mutant S662G (MUT), S662D, and S662E were incubated with purified CKIε at 0.25 pg/μl in panel A and C and at 6.25 pg/μl in panel B. The reactions were terminated at the indicated time points. In panel A and C, +phos indicates the reaction where phosphatase was added at the end of the reaction. In panel B, ‘−’ denotes that no CKIε was added, ‘+’ denotes that CKIε was added.

FIG. 8. Mapping of the CK1ε binding domain of PER2, CKIε, myc-epitope-tagged mPER2, and the indicated truncation mutants of mPER2 (lanes 1-6) were in vitro expressed in a rabbit reticulocyte lysate in the presence of 35S-methionine as previously described [Vielhaber, 2000 #941]. Lysates containing CKIε were mixed with the indicated mPER2 construct, incubated for 60 minutes at 37° C., and the mPER2 protein immunoprecipitated. The presence or absence of coimmunoprecipitating CKIε was assessed by SDS-PAGE and PhosphorImager analysis (lanes 7-11). A schematic of CKIε binding sites on mPER1 and mPER2 is shown below. The CKIε binding sites are 51% identical between the two proteins. Note the decrease in electrophoretic mobility of mPER2 fragments in the presence of CKIε, most likely due to phosphorylation by the added CKIε.

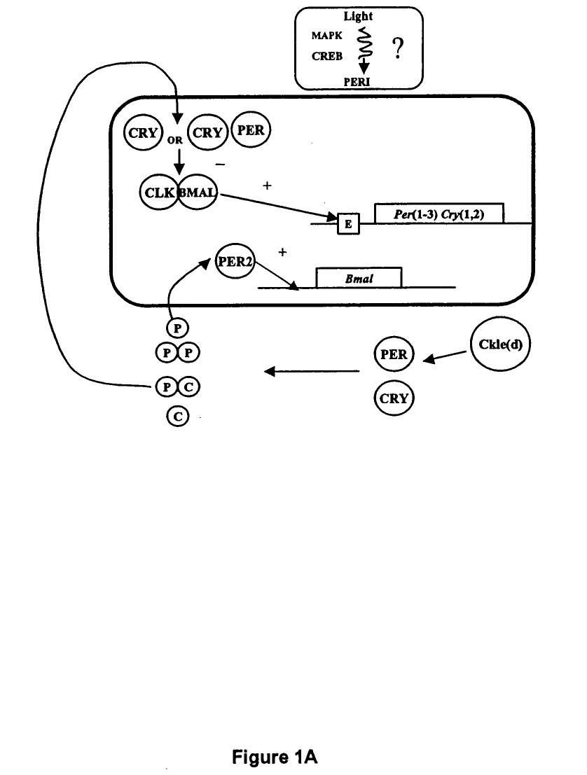

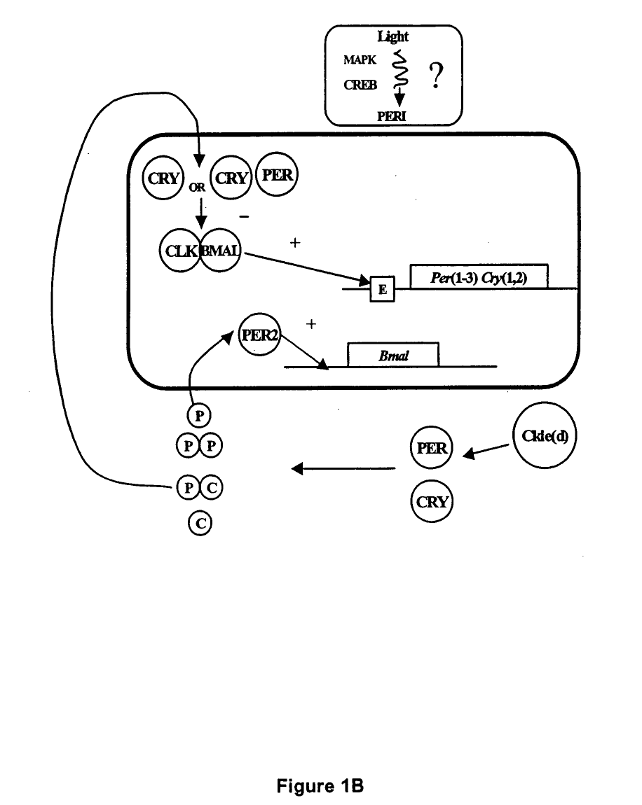

### DETAILED DESCRIPTION OF THE INVENTION

The present invention relates to the hPER2 gene and a mutant version of the hPER2 gene that participates in the human circadian biological clock. Without being limited to any one theory, it appears that the polypeptide product of the mutant hPER2 gene found in some familial advanced sleep phase syndrome patients is hypophosphorylated by casein kinase I epsilon. This appears to be due to the serine-to-glycine mutation caused by the point mutation of the genomic sequence. Without being bound to any one theory, it appears that the serine-to-glycine mutation affects the casein kinase epsilon binding region of the hPER2 protein, thus blocking the phosphorylation cascade ordinarily caused by the binding of casein kinase epsilon to hPER2

Familial advanced sleep phase syndrome is an autosomal dominant circadian rhythm variant; affected individuals are “morning larks” with a 4-hour advance of the sleep, temperature and melatonin rhythms. The localization of the FASPS gene near the telomere of chromosome 2q is reported herein. A strong candidate gene (hPer2), a human homolog of the period gene in Drosophila, maps to the same locus. Affected individuals have a serine to glycine mutation within the casein kinase Iε (CKIε) binding region of hPER2 that causes hypophosphorylation by CKIε in vitro. Thus, a variant in human sleep behavior can be attributed to a missense mutation in a clock component, hPER2, which alters the circadian period. The identification of genes influencing any aspect of human behavior is complicated by other genetic influences, behavioral tendencies, and cultural factors.

A familial abnormality of human circadian behavior that segregates in a highly penetrant autosomal dominant manner and produces a striking 4-hour advance of the daily sleep-wake rhythm was recently reported by the inventors. Jones, C. R. et al., Nat. Med. 1999, 5:1062. In those exhibiting this behavioral trait, known as familial advanced sleep phase syndrome (FASPS), sleep onset occurs at approximately 7:30 p.m., when most people are actively socializing. Sleep duration is normal, but is terminated by a spontaneous awakening at approximately 4:30 a.m. just when conventional sleepers are at their sleepiest time of the 24-hour cycle.

Biological “clocks” that free-run in constant conditions with an endogenous period (τ) close to the 24 hour period of the solar day are ubiquitous among eukaryotes and provide important adaptational advantages by anticipating the transitions between night and day. Dunlap, J. C., Cell, 1999, 96:271. The mammalian circadian pacemaker resides in the paired suprachiasmatic nuclei, and influences a multitude of biological processes including the sleep-wake rhythm. Weaver, D. R., J. Biol. Rhythms, 1998, 13:100. The core clock mechanism in the SCN interacts with other brain regions to form a circadian system that is entrained primarily by ambient light levels. Although the timing of sleep is strongly influenced by the circadian system, other factors such as social schedules, and previous sleep deprivation may predominate.

Mutagenesis screens in animals and recognition of spontaneous mutations led to the discovery of short and long τ autosomal semi-dominant circadian rhythm mutants in fungi, plants, Drosophila, and rodents. Dunlap, J. C., Cell, 1999, 96:271; Reppert, S. M., Neuron, 1998, 21: 1; Wager-Smith, K., and S. A. Kay, Nat. Genet., 2000, 26:23. Long period mutants are generally found to be phase-delayed with respect to an entraining light-dark cycle while short τ mutants are usually phase advanced. Hamblen-Coyle, M. J., et al., Journal of Insect Behavior 1992, 5:417. Genetic study of these abnormal circadian phenotypes led to the identification and characterization of clock genes responsible for circadian behavior. Lakin-Thomas, P. L., Trends Genet., 2000, 16:135. The encoded proteins function in interacting feedback loops composed of PAS domain transcription factors that are both negatively and positively controlled by regulatory phosphoproteins such as PERIOD and CRYPTOCHROME. Shearman, L. P., et al., Science 2000, 288:1013.

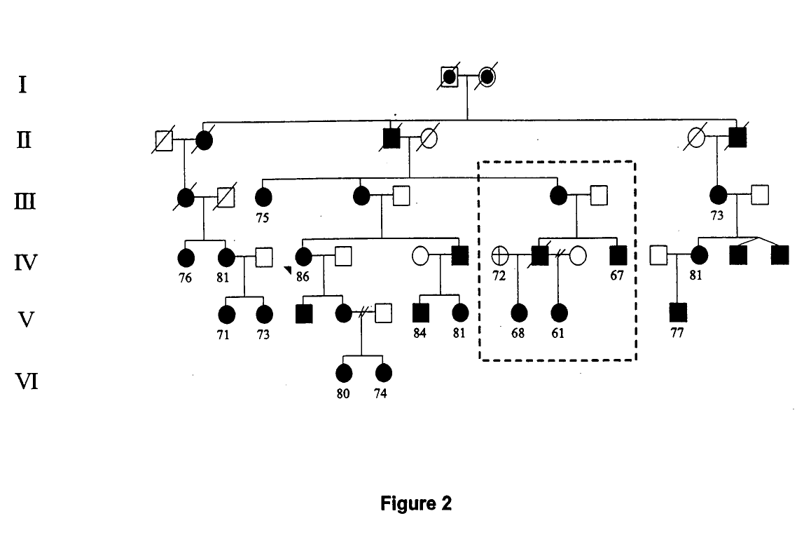

To determine the genetic basis of FASPS, linkage analysis was performed in a large family segregating an FASPS allele (K2174) (FIG. 2). Previously described strict criteria for classification of patients with FASPS were used. Jones, C. R., et al., Nat. Med., 1999, 5:1062. All participants filled out the Horne-Östberg questionnaire, a validated tool for evaluation of an individual's tendency between the extremes of “morning lark” (scores: 70-86) to “night owl” (scores: 16-30) Individuals who did not meet either the conservative affected or unaffected criteria, were classified as unknown. Venous blood samples were gathered from individuals from ASPS families who were likely to contribute to linkage information. Participants signed a “Consent of Participation” form, which was approved by the Institutional Review Board for Human Research at the University of Utah School of Medicine. High-molecular weight genomic DNA was isolated from whole-blood lysates, and lymphoblastoid cell lines were transformed with Epstein-Barr virus as described in Ptacek, L. J., et al., Cell, 1994, 77:863. In the initial automated genome-wide scan, highly polymorphic tetranucleotide and dinucleotide repeat markers, distributed every ˜20 cM across the genome, were chosen for the mapping set. The fluorescently labeled markers were used to amplify genomic DNA in total reaction volumes of 20 ml in a MJR PTC-200 thermocycler (MJ Research, Inc., Watertown, Mass.). The products were visualized on an Applied Biosystems model 377 and analyzed by the Genotyper peak-calling software. Pairwise two-point linkage analysis with MLINK of the LINKAGE program was utilized. Disease penetrance was set at 0.95, without a gender difference, and the normal and FASPS allele frequencies were set at 0.999 and 0.001, respectively. Linkage analysis revealed a number of small positive LOD scores. These were examined by PCR amplification of genomic DNA from all members of kindred 2174 with additional markers spanning these loci Manual genotyping was carried out after PCR of DNA samples with appropriate primers as previously described. Ptacek, L. J., et al., Cell, 1994, 77:863.

A maximum LOD score of ˜3 was identified for marker D8S366, but extensive genotyping of this region revealed this to be a false positive (not shown). An examination of telomeric markers for each chromosome (since the high rate of recombination at telomeres may have obscured linkage with the initial marker set) revealed a single marker, D2S395, on chromosome 2qter, which was linked to FASPS in kindred 2174 (maximum LOD score of 5.25 at θ=0.00). Simultaneously, an additional set of 400 genome-wide markers from the ABI PRISM LMS-MD10 Linkage Mapping set was used to expand the genome-wide coverage to 600 markers spaced at an average of 7 cM intervals. D2S 125, the marker in this set nearest to D2S395 had a maximum LOD score of 1.75 at θ=0.10, but analysis of this data set did not reveal any other loci with significant LOD scores. Manual linkage analysis was also performed with 7 additional markers previously localized to a 19 cM region of chromosome 2qter Markers D2S338, D2S2338, D2S2285, D2S2253, D2S125, D2S 395, D2S 140, D2S2986, and D2S2987 (from centromere to telomere) were used for genotyping and haplotype analysis; C. Dib et al., Nature 380, 152 (1996). Evaluation of K2174 with the additional markers yielded a maximum LOD score of 3.81 at θ=0.05. For each of these markers, it was found that individuals initially classified as affected in branch 3 carried a different allele than the one segregating with ASPS in the rest of the family (FIG. 2). The haplotype generated using these markers cosegregated with ASPS in all affected individuals of K2174 except those in branch 3.

A homolog of the Drosophila period gene hPer2 resides on chromosome 2qter and is an excellent candidate gene for FASPS. Of the three human period homologs, hPer2 is the most similar to dper. Albrecht, U., et al., Cell, 1997, 91:1055. In addition, mutations in Per in the fly and in the mouse produce a similar short period phenotype. Konopka, R. J., and S. Benzer, Proc. Natl. Acad. Sci. U.S.A. 1971, 68:2112; and Zheng, B., et al., Nature, 1999, 400:169. In humans (and other animals), short period mutations are predicted to phase advance circadian rhythms under entrained conditions. Klerman, E. B., et al., Am, J. Physiol., 1996, 270:R271; J. Duffy et al., Sleep, 1999, 22:S92. Furthermore, unlike mPer1 and mPer3, the phase response curve for light induction of mPer2 RNA is maximal at CT 14 when phase delays are elicited by light. Zylka, M. J., et al., Neuron 1998, 20:1103. This is consistent with a predominantly phase delay function for mPER2. Thus, a loss-of-function mutation in hPER2 could, theoretically, lead to a phase advance.

The localization of hPer2 on chromosome 2qter was confirmed by isolating a BAC clone (552H8, CITB human BAC library) containing the hPer2 gene for use in fluorescence in situ hybridization experiments.

In these, human lymphoblast cultures were treated with 0.025 mg/ml cholcimid at 37° C. for 1.5 hr. Cholcimid treated cultures were pelleted at 500×g at room temperature for 8 min. The resulting pellets were then re-suspended with 0.075M KCl, 3 ml per pellet, for 15 minutes at room temperature. The cells where then fixed in 3:1 MeOH:acetic acid and stored at 4° C. The human BACs were labeled with spectrum orange using a nick translation kit per the manufacturers protocol (Vysis, Downers Grove, Ill.). Slides were then prepared by dropping fixed cells onto glass slides and washing with excess fixative. The slides were then washed in acetic acid for 35 min. at room temperature and dehydrated in 70%, 85%, and finally 100% EtOH (at 2 min each). Chromosomes were denatured in 70% formamide in 2×SSC at 74° C. for 5 minutes, and slides were dehydrated again as above, except in ice-cold EtOH.

Two mg of labeled probe was then blocked with 2 mg of human Cot-1 DNA in Hybrisol VI (ONCOR, Gaithersbug, Md.). The probe mixture was denatured at 74° C. for 5 minutes and then pre-annealed at 37° C. for 15 min. Twelve ml of pre-annealed probe was applied per slide, a cover slip was added, and edges were sealed with rubber cement. The slides were then hybridized. Following this, the slides were washed in 0.4×SSC containing 0.1% Tween-20 at 74° C. for 2 min., followed by 1 min. at room temperature in 2×SSC. The prepared slides were allowed to dry in the dark at room temperature and were stained with DAPI (Vector labs, Burlingame, Calif.) for chromosome visualization.

The BAC mapped to the tip of chromosome 2q (not shown). A polymorphism in hPer2 was used to genotype K2174 and to perform two-point linkage mapping with 9 markers noted previously. Markers D2S338, D2S2338, D2S2285, D2S2253, D2S125, D2S395, D2S140, D2S2986, and D2S2987 (from centromere to telomere) were used for genotyping and haplotype analysis. A recombination demonstrated the hPer2 gene to be distal to marker D2S338. The haplotype of the remaining 8 markers was fully linked to hPer2 in this family. The individuals in branch 3 were considered to represent phenocopies and mutation analysis of hPer2 was performed.

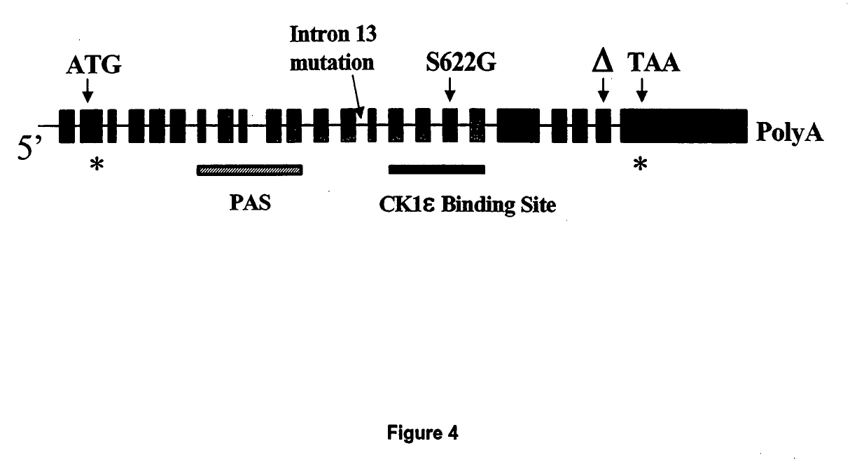

As characterized, human Per2 comprises 23 exons (see FIG. 4). The hPer2 intron/exon boundaries were determined in order to carry out the mutational analysis. Intron/exon boundaries of the hPer2 gene were obtained by a combination of direct sequencing of hPer2 BAC DNA, and sequencing of PCR products from genomic DNA with primers distributed along the entire cDNA. At least 100 base pairs of intronic sequence flanking each exon boundary were obtained. Intron sizes were determined directly from genomic sequence or estimated by the size of PCR products amplified using oligonucleotides from adjacent exons. All sequencing reactions were carried out with an Applied BioSystems model 377 DNA sequencer (Foster City, Calif.). A sequencing error of the hPer2 cDNA (Genbank accession #NM 003894) was identified; the reported cDNA has a missing base at position 3652 that shifts the reading frame, predicting translation of 69 amino acids that are not homologous to other PER proteins before the stop codon. With the corrected sequence, the region 3′ of that base encodes 78 amino acids that are 64% identical to mPER2.

Single-strand conformation polymorphism analysis (“SSCP” was carried out as described in Ptacek, L. J., et al., Cell 1994, 77:863. PCR products were diluted, denatured, and electrophoresced through acrylamide gels and visualized on X-ray film at ˜80 C for 12-24 hours. Aberrant SSCP bands were cut directly from the dried gels and sequenced as described in L. J. Ptacek et al., Cell 1994, 77:863. Those of affected and unaffected individuals revealed a complex banding pattern in exon 17. Sequencing of this exon from individuals in K2174 revealed four changes. Three of the four changes (bp2087 A/G, bp2114 A/G, and bp2117 A/G) occur at wobble positions and therefore preserved the amino acid sequence. However, the base change at position 2106 (A to G) of the hPer2 cDNA predicts substitution of a serine at amino acid 662 with a glycine (S662G) (FIG. 4). This change was not found in 92 controls. The S662G change co-segregates with the ASPS phenotype in this family except for the branch in which the FASPS-associated marker alleles were unlinked (FIG. 2). Four additional at-risk individuals in the family carry the mutation but did not meet strict affection criteria, although they showed a strong tendency of early sleep-wake preference (Horne-Östberg scores 74.4±7.2, n=4). Jones, C. R., et al., Nat. Med., 1999, 5:1062.

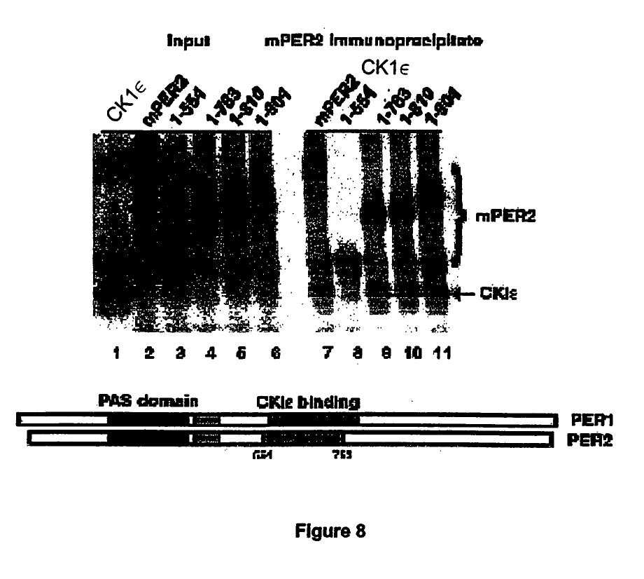

To establish whether this mutation causes FASPS, the S662G mutation was functionally characterized. First, cDNA clones encoding mPer 2 and hPer 2 were PCR amplified from the corresponding plasmids and cloned into the pCS2+MT vector as previously described in Vielhaber, E., et al., Mol. Cell. Biol. 2000, 20:4888. Site-directed mutagenesis of the serine residue at position 662 of hPER2 and the analogous serine (659) of mPER2 were performed to substitute a glycine residue. Mutagenesis was carried out with the QuikChange™ Site-directed Mutagenesis Kit (Strategene) using the protocol outlined therein. EcoRI-Xba1 fragments encoding amino acids 474 to 815 of hPER2 (and the corresponding amino acids 472 to 804 of mPER2) were PCR-amplified with primers containing EcoRI and Xbal sites, gel-purified with the GENECLEAN kit (BIO 101) and directionally cloned into the EcoRI-Xbal sites of the pCS2+MT vector. Expression from an SP6 promoter generates 6-myc-tagged peptides. A series of 3′ deletion mutations of mPer2 were constructed (encoding amino acids 1-554, 1-763, 1-810, and 1-904) for use in mapping the binding site for CKIE as previously described for mPer1. Vielhaber, E., et al., Mol. Cell. Biol. 2000, 20:4888. All constructs were confirmed by sequencing. To determine whether S662 is located within the CKIε binding site of hPER2, CKIε, myc-epitope-tagged mPER2, and the indicated truncation mutants of mPER2 (FIG. 8) were expressed in rabbit reticulocyte lysates and mPER2 peptides were immunoprecipitated with antibodies to myc. Vielhaber, E., et al., Mol. Cell. Biol. 2000, 20:4888. CKIε was co-precipitated with mPER2 (1-763) but not mPER2 (1-554), thus demonstrating that the CKIε binding site of mPER2 is located between residues 554 and 763 corresponding to residues 556 to 771 of hPER2 (FIG. 8).

Studies of doubletime mutants in Drosophila and the tau mutant in the golden hamster indicate that mutations affecting the function of CKIε disrupt endogenous circadian clock function leading to altered period lengths or arrhythmicity. Kloss, B., et al., Cell, 1998, 94:97; Lowrey, P. L, et al., Science, 2000, 288:483. In addition, hPER2 and mPER2 are substrates of CKIε. Since the S662G mutation is located within the CKIε binding region, hPER2 and mPER2 fragments extending from amino acids 474 to 815 and 472 to 804, respectively, were used to evaluate the effect of the mutation on PER2 phosphorylation. Transcription and translation of hPer2 and mPer2 inserts were performed in vitro in the presence of 35S-methionine with the TnT SP6 Coupled Reticulocyte Lysate System (Promega) over a period of 90 minutes at 30° C. The labeled products were incubated with CKIε in buffer containing phosphatase inhibitors (25 mM Tris HCl, pH 7.5, 15% glycerol, 20 mM NaF, 170 nM okadaic acid, 2 mM dithiotreitol [DTT], 10 mM β-glycerol phosphate and 150 μM ATP). 20 μL aliquots were removed at selected timepoints and boiled with SDS gel-loading buffer (0.1% bromophenol blue, 50 mM Tris HCl, pH 6.8, 0.1 M DTT, 2% SDS, 10% glycerol) to stop the reaction. At the end of the experiment, 20 μL aliquots were digested with 35 units of calf intestinal phosphatase in buffer (50 mM Tris HCl, pH 7.9, 10 mM MgCl₂, 0.1M NaCl, 1 mM DTT) for 30 minutes where indicated. All products were analyzed by electrophoresis in 8% SDS-PAGE gels with an acrylamide:bis-acrylamide ratio of 120:1 to enhance mobility shifts. The gels were fixed and dried and the bands visualized using PhosphorImager screens scanned with Scanner Control SI software (Molecular Dynamics, Sunnyvale, Calif.).

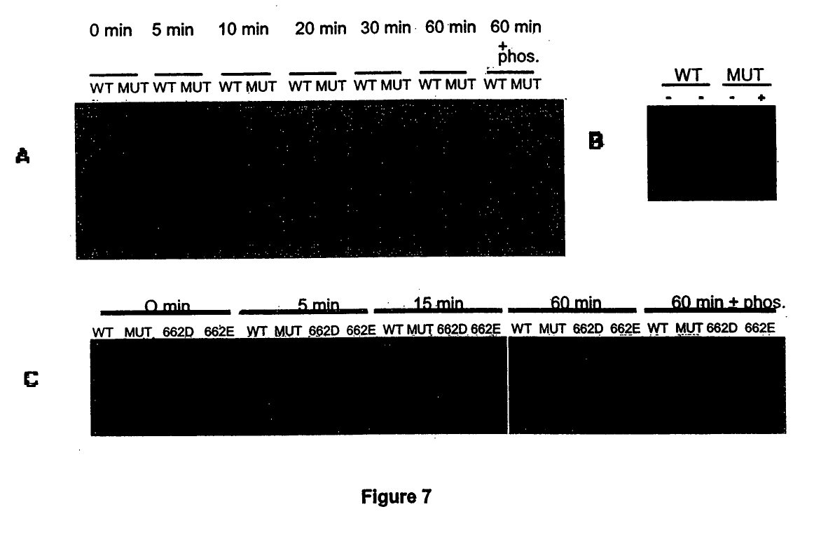

To test whether the S662G mutation eliminates a potential phosphorylation site, reticulocyte lysates containing 35S-labeled hPER2 fragments were incubated with a low concentration of CKIε (0.25 ng/μl). An electrophoretic mobility shift was observed when wild-type (S662), but not mutant (G662), fragments were treated with CKIε (FIG. 7A). A similar result was obtained with wild-type and mutant mPER2 fragments (not shown). Phosphatase treatment confirmed that this shift was due to phosphorylation (FIGS. 7A, 7C). When the experiment was repeated with a higher concentration of CKI ε (6.5 ng/μl), mobility shifts were observed for both the wild type and mutant hPER2 fragments (FIG. 7B). Thus, regardless of the phosphorylation status of S662, other residues in the peptide can be phosphorylated with excess kinase.

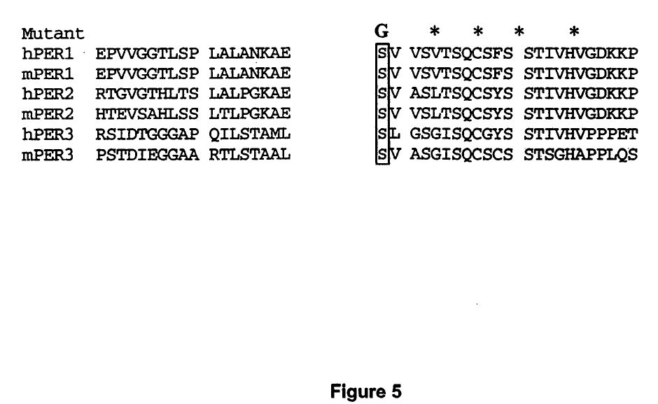

CKIε preferentially phosphorylates peptides with acidic [for example, DDDD-X-X-S] or phosphorylated residues [for example, S(P)-X-X-S] immediately upstream of the target residue (where D is aspartate, S is serine, S(P) is a phosphoserine, X is any amino acid, and the underlined ‘S’ is the target of the subsequent phosphorylation). Flotow, H., et al., J. Biol. Chem., 1990, 265:14264; Cegielska, A., et al., J. Virol., 1994, 68:269. Analysis of the hPER2 sequence reveals 4 additional serine residues, carboxy-terminal to S662, that follow the pattern S_X_X_S (FIG. 5). It was speculated that after S662 is phosphorylated, it would create a CKIε recognition site facilitating the phosphorylation of S665, and so on. This entire series of serines could therefore be modified by CKIε after S662 is phosphorylated in a cascade of subsequent phosphorylations as described previously for phosphorylation of p53 by CKI (FIG. 5). Dumaz, N., D. M. Milne, and D. W. Meek, FEBS Lett., 1999, 463:312; Sakaguchi, K., et al., J. Biol. Chem., 2000, 275:9278. Such screens could be conducted with the addition of a compound to test the properties of the compound for inhibition or upregulation of the phosphorylation of hPER2 by casein kinase epsilon. Potential inhibitors could be tested in the presence of casein kinase epsilon, hPER2, and phosphates, with measurements of phosphorylation levels showing lower phosphorylation than in a reaction without the potential inhibitor shows an inhibitor. Additionally, potential upregulators would cause an increase in phosphorylation levels over hPER2 exposed to casein kinase epsilon and phosphates alone.

To test this idea further, the serine residue at position 662 was mutated to aspartate, reasoning that the presence of a negative charge from the acidic residue would mimic a phosphoserine. Supporting this hypothesis, the CKIε-dependent phosphorylation was restored in the S662D mutant (FIG. 5). At levels of CKIε that were not sufficient to cause a mobility shift in the S662G protein, both wild type and S662D hPER2 had robust mobility shifts. Therefore, phosphorylation of S662 may regulate the subsequent phosphorylation of a series of downstream residues.

Interactions between PER2 and CKIε also provide a strong rationale for hPer2 being involved in the molecular pathogenesis of FASPS. In a current mammalian clock model, mPER2 is a positive regulator of the Bmal1 feedback loop, raising the possibility that phase advance of hPer2 could phase advance the feedback loop. Shearman, L. P., et al., Science, 2000, 288:1013. A semidominant mutation in CKIε was recently shown to be responsible for the advanced sleep phase and short τ in the tau mutant Syrian hamster. Lowrey, P. L., et al., Science, 2000, 288:483. The point mutation R178C substitutes cysteine for arginine in an anion-binding pocket on the structure of the kinase, potentially decreasing the ability of the kinase to recognize acidic or phosphorylated residues that define the CKI recognition motif. Lowrey, P. L., et al., Science 2000, 288:483. Thus, the tau mutation may decrease phosphorylation of PER residues downstream of S662 due to diminished recognition of phosphoserine 662, while the FASPS mutation S662G mirrors this effect by preventing phosphorylation of residue 662.

Taken together, the tau mutant CKIε and the FASPS mutation in hPER2 suggest that one critical function of CKIε is to phosphorylate hPER2. Phosphorylation of PER by CKI may promote its degradation during the circadian cycle. Vielhaber, E., et al., Mol. Cell. Biol., 2000, 20:4888; Kloss, B., et al., Cell, 1998, 94:97; Price, J. L., et al., Cell, 1998, 94:83; and Keesler, G. A., et al., Neuroreport, 2000, 11:951. Deficient phosphorylation of hPER2 in the cytoplasm could impair its degradation and/or accelerate its nuclear entry and thus hasten its accumulation. This would phase advance the rhythm of hPer2, perhaps in part by increasing transcription of Bmal 1 and repress transcription of the Per genes. The net result might be a shortening of τ and an advance of the sleep-wake rhythm as seen in FASPS.

Human casein kinase Iδ (“hCKIδ”) is the closest homologue to human casein kinase I ε (“hCKIε”), and associates with and phosphorylates hPER1, thus similarly causing protein instability. It has also been observed that both hCKIδ and hCKIε phosphorylated and caused protein instability of human period 2 protein (hPER2). Immunohistochemical staining of rat brains demonstrates that CKIδ protein is localized in the suprachiasmatic nuclei, the central location of the master clock, as discussed above. Without being bound to any one theory, these results indicate that CKIδ likely plays a role similar to that of CKIε, suggesting that it may also be involved in regulating circadian rhythmicity by post-translation modification of mammalian clock proteins hPER1 and hPER2. Camacho et al., Human casein kinase Iδ phosphorylation of human circadian clock proteins period 1 and 2, FEBS Letters, (2001) 489:159-165.

Of the seven identified CKI isoforms, CKIδ is the closest homologue of CKIε. The kinase domains of both of these proteins are 97% identical with only eight amino acid changes. Graves, P. R. et al., J. Biol. Chem., (1993), 268:6394-6401; and Fish et al., J. Biol. Chem., (1998), 270:14875-14883. While the identification of mutant CKIε in the tau mutant hamster suggests CKIε is the important circadian regulator, an alternative hypothesis is that the tau mutation renders CKIε a dominant interfering kinase that may block the function of CKIε and CKIδ in the SCN. It has been shown that hCKIδ was capable of playing a similar role as hCKIε in the phosphorylation of human clock proteins PER1 and 2.

Recombinant hCKIδ or hCKIε have been shown to phosphorylate hPER1 and hPER2 in transfected cells. Camacho, F., et al., Human casein kinase Iδ phosphorylation of human circadian clock proteins period 1 and 2, FEBS Letters, (2001) 489:159-165. In the Comacho et al. study, HEK 293T cells were transfected with vector alone or with hPER1 or hPER2 (lanes 1, 2, and 5), hPER1 and hCKIδ (lane 4), hPER2 and hCKIδ (lane 7), or HPER1 and hCKIε (lane 3), or hPER2 and hCKIε (lane 6) and a Western blot analysis of hPER1, hPER2, hCKIδ, and hCKIε was conducted. At 16 hours post-transfection, cells were harvested and lysates were prepared. 20 mg of total protein from the HEK 293T lysates obtained was loaded onto a 3-8% gradient SDS-PAGE. Proteins were transferred to PVDF membranes and Western-blotted using the anti-YFP mAb (1:1000), or anti-c-myc (hCKIδ) mAb (1:1000), or anti-HA (hCKIε) mAb (1:1000). Camacho, F., et al., Human casein kinase Iδ phosphorylation of human circadian clock proteins period 1 and 2, FEBS Letters, (2001) 489:159-165.

Study of other FASPS families in an available database demonstrated that some are unlinked to the hPer2 locus, thus establishing the existence of locus heterogeneity in FASPS (not shown). Additional hPer2 mutations in other ASPS probands were not identified. It is possible that mutations in intronic DNA have been missed that lead to alterations of hPer2 expression. Short-period animal models caused by mutations in other genes, along with a failure to find other hPer2 mutations in FASPS kindreds, predict that additional FASPS genes remain to be identified.

The following lines of evidence support the conclusion that the S662G mutation is responsible for FASPS in this family: 1) the FASPS allele in K2174 is linked to chromosome 2qter with significant LOD scores despite the recombinant branch; 2) hPer2 is a physiologically relevant gene on chromosome 2qter and harbors the S662G mutation in all affected and genetically linked individuals; 3) genome-wide linkage analysis with 600 markers spaced at average intervals of 7 centimorgans did not identify another linked locus; 4) the S662G mutation was not found in a large number of control chromosomes; and 5) the mutation leads to decreased phosphorylation by a kinase (CKIε) that, when mutated, causes a similar phenotype in Drosophila and the golden hamster. Taken together, these data demonstrate that hPer2 is an ortholog of the dper gene and is a physiologically relevant target of CKIε, providing the first direct link between human clocks and those of model systems. The ASPS individuals in branch 3 did not carry the S662G mutation and therefore represent phenocopies of the ASPS phenotype (Science Online).

The recognition that Mendelian circadian rhythm mutations occur in humans predicts that the elements of the human clock can now be systematically dissected. Other families in which an FASPS allele is not co-segregating with hPer2 will provide an opportunity to identify mutations in other genes that lead to alterations of human circadian rhythms. Such discoveries will likely provide novel insights into human sleep and may ultimately improve the ability to treat not only ASPS, but also other sleep-phase disorders such as sleep-phase delay, ASPS of aging, jet-lag, and shift work.

It has subsequently been shown that serine 662 is a substrate for phosphorylation and that phosphorylation at this residue makes the hPer2 a better substrate for phosphorylation downstream by casein kinase 1ε. Using peptides in this region it has been shown that the threonine and tyrosine residues in the region are not phosphorylated by casein kinase 1ε, but multiple serines are phosphorylated by casein kinase 1ε once the serine at position 662 has a negative charge (either a covalently linked phosphate during synthesis of the peptide or replacement of this serine with an aspartate). Both the biochemical phosphorylation assay and mass spectrometry support the phosphorylation of an additional four moles per mole of substrate when there is a negative charge at serine 662.

Interestingly, this motif (sxxsxxsxxsxxs) is present in a number of proteins in the databases including the adenomatous polyposis coli protein and multiple members of the [groucho]-like family that are co-repressors of WNT signaling. The mutation at serine 662 apparently leads to hypophosphorylation of per2 which may lead to more stable protein that accumulates faster thus shortening the period of the clock in individuals carrying this genetic variant. It is noteworthy that phosphorylation at the initial serine residue leads to a very rapid phosphorylation of subsequent residues in what appears to be an all-or-none switch. This may be a common motif in regulation of proteins by casein kinase 1 phosphorylation.

Another interesting observation during the cloning of the hPer2 gene is that it appears to be one of the last (if not the last) gene on chromosome 2q. It is well known that telomeres are foci for heterochromatin accumulation over time. Heterochromatin is a structure that is not fully understood but that involves DNA wrapped around a histone octomer along with a number of other proteins. The presence of heterochromatin can interfere with the ability of transcriptional machinery to access genes thereby repressing their transcription. It is not required that the gene be on the telomere to experience such transcriptional modifications but the position on the chromosome in this case hinted at this being a possibility whereby, with aging, heterochromatin may accumulate in some individuals to the point that hPer2 transcription is reduced. Thus, without being limited to any one theory, it appears that the ASPS of aging, a phenomenon that has widely been thought to result as a normal part of aging, may in fact be the result (at least in part) of transcriptional repression of hPer2 by heterochromatin on the telomere of chromosome 2q.

### EXAMPLES

### Example 1

### Discovery of and Characterization of an FASPS Family

The first ASPS patient presented to a sleep center with disabling early evening sleepiness and early morning awakening. Because the patient recognized a similar trait in some family members, consenting relatives from her extended family were evaluated. A structured interview with each participant focused on the underlying preferred sleep schedule in the absence of psychosocial factors that would delay or advance sleep phase. Individuals were considered ‘affected’ if they described a life-long, stable pattern of early sleep onset and offset and met strict classification criteria. Jones, C. R., et al., Familial advanced sleep-phase syndrome. A short-period circadian rhythm variant in humans, Nat. Med., 1999, 5(9):1062-5. The Horne-Östberg questionnaire, a validated measure of “morning lark” vs. “night owl” tendency, was administered to each subject. Horne, J. A. and O. Östberg, A self-assessment questionnaire to determine morningness-Eveningness in human circadian rhythms, International Journal of Chronobiology, 1976,4:97-110.

Using strict classification criteria, 29 people with FASPS and 46 unaffected people were identified. ASPS appears to segregate as a highly penetrant autosomal dominant trait in this family (FIG. 2). This family is of Northern European descent. The youngest affected subject was eight years old. Most FASPS subjects knew they were obligate “morning larks” by the age of thirty. Horne-Östberg scores were consistent with a selected classification scheme {FASPS [76.5±6.0 (n=12)] vs. unaffected relatives [60.7±7.2 (n=11)] (p<0.0005)}. Id.

### Example 2

### A Study of Physiologic Parameters of FASPS Individuals

Six FASPS subjects (ages 20-69, average 37±18 years) were gender- and age-matched (±6 years) to six unrelated controls on conventional sleep-wake schedules for an inpatient study. All participants underwent a medical history and physical exam and were found to be generally healthy. The Beck Depression Inventory (BDI) was administered with scores from both FASPS and control groups falling into the range of “minimal depression.” Beck, A. T., The Beck Depression Inventory, 1978, The Psychological Corporation: Harcourt Brace Jovanovich.

The 12 inpatient subjects were admitted in the early afternoon for two consecutive nights of polysomnographic (PSG) assessment of sleep phase and sleep quality followed each morning by a Multiple Sleep Latency Test (MSLT). The MSLT estimates sleepiness by measuring the latency to sleep onset in multiple nap trials during the day. The PSG and MSLT recordings were performed and scored according to standard procedures. Keenan, S. A., Polysomnographic Technique: An overview, in Sleep Disorders Medicine: Basic Science, Technical Considerations and clinical Aspects, 1994, Butterworth-Heinemann: Boston, p. 79-94; Carskadon, M. A. and W. C. Dement, Normal human sleep: An overview, Second ed., Principles and practice of sleep medicine, 1994, Philadelphia: W. B. Saunders Company, 16-25; Rechtschaffen, A. and A. Kales, A manual of standardized terminology, techniques and scoring system for sleep stages of human subjects. 1968, Los Angeles: UCLA Brain Information Service/Brain Research Institute. No common sedatives or stimulants were detected in the urine of subjects after the first night of PSG recording. Both the PSG measures of sleep at night and the MSLT values during the day were similar on days 1 and 2 of recording and were therefore averaged.

Polysomnographic measures of sleep phase including the time of sleep onset, sleep offset, first slow wave sleep and first REM sleep were advanced by almost 4 hours in FASPS subjects compared to controls (see Table 1). Other confounding factors that might explain this phase advance were not seen. Specifically, there was no consistent seasonal bias for date of inpatient study in FASPS vs. control subjects and no evidence of significant voluntary sleep restriction. PSG measures of sleep quality and quantity were within normal limits for both FASPS and control groups [FASPS (n=5) vs. Control (n=6): Total Sleep Time (minutes); 425.3±59.92 vs. 445.42±83.48; % Stage 1 Sleep; 11.72±3.79 vs. 12.83±4.37; % REM Sleep; 20.08±3.72 vs. 21.00±7.58; % Slow Wave Sleep; 10.30±7.02 vs. 10.44±3.59]. One FASPS subject had evidence of moderate obstructive sleep apnea and one control had periodic limb movements in sleep with micro-arousals. None of the MSLT results from control or FASPS subjects were suggestive of narcolepsy or other cause of excessive daytime sleepiness.

TABLE 1
Phase Markers of Overt Rhythms

| | Control (n = 6) | FASPS (n = 6) | Difference | |
|---|---|---|---|---|
| | Mean ± SD | Mean ± SD | (hours:minutes) | P value |
| Sleep | 23:10 ± 0:40 | 19:25 ± 1:44 | 3:45 | <.0005 |
| Onset | | | | |
| Sleep | 07:44 ± 1:13 | 04:18 ± 2:00 | 3:26 | <.0005 |
| Offset* | | | | |
| 1st Slow | 23:55 ± 1:17 | 20:14 ± 2:35 | 3:41 | .002 |
| Wave | | | | |
| Sleep | | | | |
| 1st REM* | 00:55 ± 1:29 | 21:16 ± 2:25 | 3:39 | <.0005 |
| DLMO | 21:21 ± 0:28 | 17:31 ± 1:49 | 3:50 | <.0005 |
| Temp | 03:35 ± 1:33 | 23:22 ± 2:55 | 4:13 | .002 |
| Nadir** | | | | |

*n = 5 for FASPS only:
**n = 5 for control and FASPS. Data (when available) include both nights of study.

Circadian phase was determined using plasma melatonin and body core temperature measurements as discussed in Lewy, A. J. & R. L. Sack, The dim light melatonin onset as a marker for circadian phase position, Chronobiol. Int., 1989, 6(1):93-102. As seen in Table 1, the melatonin and temperature rhythms were both phase advanced by 3-4 hours in FASPS subjects relative to controls. To control for possible sleep deprivation or self-imposed unconventional sleep/wake schedules, sleep logs were kept at home for one week before admission and for two weeks after leaving the Clinical Research Center. Activity levels (actigraphy) were also recorded during the inpatient stay and for three weeks after going home. The phase advance of self-reported sleep times in FASPS vs controls was consistent with ambulatory actigraphy and sleep log data. By all three measures, FASPS individuals were sleep-phase advanced by 3 to 4 hours compared to the controls (data not shown). On vacation, this group of FASPS subjects went to sleep between 5:00 and 8:00 p.m. (18:58±1:03, n=6) and woke up between 1:00 and 4:30 a.m. (3:13±1:24, n=6). The large difference in Horne-Östberg scores for FASPS vs. control individuals [77±6.7 (n=5) vs. 48.2±4.6 (n=6) (p=0.006)] is consistent with a phase advance of this large magnitude. The average Horne-Östberg score of 48.2 for the controls also supports the conclusions of the sleep log, actigraphy and clinical assessment that control subjects were not sleep-phase delayed.

### Example 3

### Free Running Period Measurement

One 69-year-old subject was studied in a time isolation facility to determine the intrinsic period of her circadian clock. Immediately following a three-day entrainment period on a 24-hour sleep/wake schedule, she was studied for 18 days in a laboratory apartment without any cues to time of day. The subject was instructed to eat and sleep whenever she felt inclined, with the exception that she was requested not to take naps. During her waking hours, the subject was permitted to carry out leisure activities in 150-lux ambient light. EEG and body core temperature were recorded continuously throughout her 3-week laboratory stay. The sleep and wakefulness scoring by standard criteria demonstrated normal sleep architecture and sleep quality. Rechtschaffen, A. and A. Kales, A manual of standardized terminology, techniques and scoring system for sleep stages of human subjects, 1968, Los Angeles: UCLA Brain Information Service/Brain Research Institute. Periodograms revealed a remarkably short τ (23.3 hours) for both rhythms compared to a gender and age-matched control (24.2 hours) and to estimates of 24.0 to 24.5 hours in other studies Jones, C. R., et al., Familial advanced sleep-phase syndrome. A short-period circadian rhythm variant in humans, Nat. Med., 1999, 5(9):1062-5; Campbell, S. S., D. Dawson, and J. Zulley, When the human circadian system is caught napping: evidence for endogenous rhythms close to 24 hours, Sleep, 1993, 16(7):638-40.

### Example 4

### Identification, Clinical Evaluation, and Collection of Additional FASPS Probands and Families

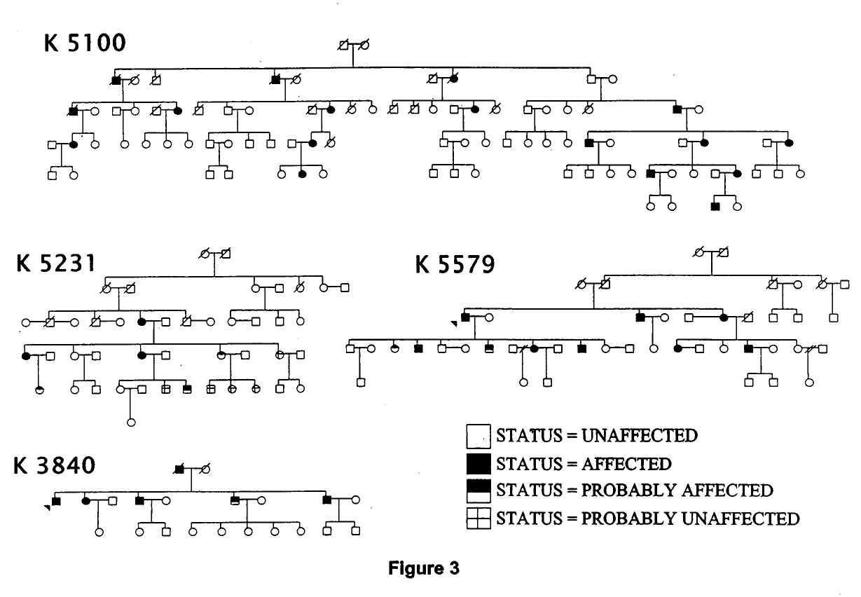

Initially, kindred 2174 and two additional smaller FASPS pedigrees were identified. When these were reported, the publicity that followed led to many calls, letters and emails from individuals who felt they were similarly affected. Jones, C. R., et al., Familial advanced sleep-phase syndrome: A short-period circadian rhythm variant in humans, Nat. Med., 1999, 5(9):1062-5. As expected, many of these did not meet the strict affection criteria adopted for the study. However, some of them did. In the last year, an additional 21 ASPS probands were identified. Evaluation of these probands and their families using the above-noted criteria revealed that a majority of these had a family history suggestive of autosomal dominant transmission. Jones, C. R., et al., Familial advanced sleep-phase syndrome: A short-period circadian rhythm variant in humans, Nat. Med., 1999, 5(9):1062-5. Some of these families are shown in FIG. 3. To date, DNA has been collected from 170 individuals in these families (57 “affected”, 19 “probably affected”, 8 “probably unaffected, 55 “unaffected” and 31 “unknown”.

### Example 5

### Mapping of the FASPS locus in Kindred 2174

The initial genome-wide scan revealed a number of small positive LOD scores which were further investigated by genotyping additional flanking markers in the family. A maximum lod score of ˜3 was identified for marker D8S366, but extensive genotyping of this region revealed this to be a false positive (data not shown). Distal telomeric markers for each chromosome were next carefully examined since the high rate of recombination at telomeres may have obscured linkage with the initial marker set. In this screen, a single marker, D2S395, was found on chromosome 2qter, which was linked to FASPS in kindred 2174 (maximum LOD score of 5.25 at θ=0.0). Simultaneously, an additional set of 400 genome-wide markers from the ABI PRISM® LMS-MD10 Linkage Mapping set was used to expand the genome-wide coverage to 600 markers spaced at an average of 5-10 cM intervals. D2S125, the marker in this set nearest to D2S395, had a maximum LOD score of 1.75 at θ=0.1. Otherwise, analysis of this data set did not reveal any other loci with significant LOD scores. Linkage data for a dense array of markers on chromosome 2q is shown in Table 2.

TABLE 2
LOD scores for chromosome 2qter markers in kindred 2174.

| | Recombination fraction (θ) | | | | | | |
|---|---|---|---|---|---|---|---|
| Marker | 0 | 0.01 | 0.05 | 0.1 | 0.2 | 0.3 | 0.4 |
| D2S338 | −0.64 | 1.30 | 1.76 | 1.75 | 1.34 | 0.81 | 0.34 |
| D2S2338 | 2.23 | 2.19 | 2.03 | 1.82 | 1.36 | 0.91 | 0.46 |
| D2S2285 | −1.60 | 0.58 | 1.09 | 1.13 | 0.89 | 0.52 | 0.21 |
| D2S125 | −0.07 | 1.07 | 1.64 | 1.75 | 1.55 | 1.12 | 0.59 |
| D2S395 | 5.25 | 5.17 | 4.83 | 4.36 | 3.29 | 2.11 | 0.93 |
| D2S140 | −0.19 | 2.32 | 2.70 | 2.57 | 1.95 | 1.18 | 0.47 |
| D2S2987 | −0.63 | 0.24 | 0.84 | 0.98 | 0.88 | 0.63 | 0.32 |

Markers are arranged from centromere at top to telomere at bottom.

### Example 6

### The Mapping of hPer2 to the FASPS1 Locus and its Mutation in Affected Individuals

A limited amount of genomic sequence in the database suggested that a homologue of the Drosophila period gene (hPer2) resides on chromosome 2qter. This was confirmed by isolating a BAC clone (RP11-7908) containing the hPer2 gene and used it in fluorescence in situ hybridization (FISH) experiments. This BAC mapped to the tip of chromosome 2q by FISH (data not shown).

Genomic structure analysis revealed that hPer2 is comprised of 23 exons (FIG. 4). A sequencing error of the hPer2 cDNA (Genbank accession #NM 003894) was identified through analysis of the genomic sequence. The reported cDNA has a missing base at position 3652 that shifts the reading frame, predicting translation of 69 amino acids that are not homologous to other PER proteins before the stop codon. With the corrected sequence, the region 3′ of that base encodes 78 amino acids that are 64% identical to mPER2.

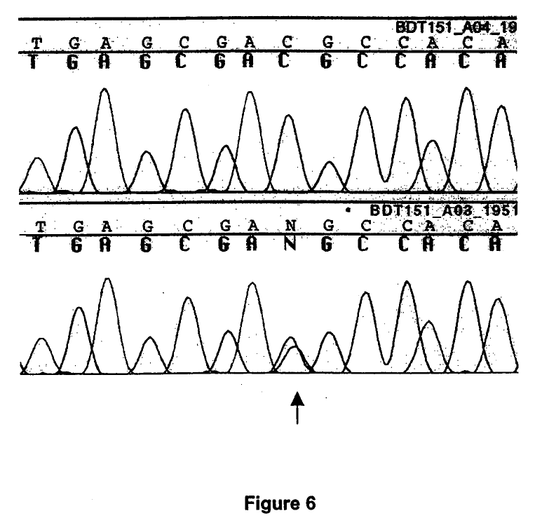

SSCP was performed on DNA from two affected individuals in K2174 and one proband from each of the other families. The hPer2 gene was completely sequenced in two individuals from K2174 since this is the only family known to be linked to chromosome 2qter. A complex banding pattern was found during SSCP analysis of exon 17. Sequencing of this exon in FASPS DNAs from K2174 revealed four changes. Three of the four changes are bp2087 A/G, bp2114 A/G and bp2117 A/G. These changes occur at wobble positions and therefore preserved the amino acid sequence. However, the base change at 2106bp (A to G, FIG. 6) of the hPer2 cDNA predicts substitution of a highly conserved serine at amino acid 662 with a glycine (S662G) (FIG. 6). In 92 normal controls, no change at this position was found. The S662G change co-segregates with the ASPS phenotype in this family.

A base change (C to T) was identified in intron 13 in one of 23 probands unrelated to K2174. This change is located 18 bases from the exon/intron boundary. SSCP of 138 control samples (276 chromosomes) showed no change at this position suggesting this alteration to be either a mutation or a very rare polymorphism. Reverse transcription of mRNA from transformed lymphoblasts from this individual, followed by PCR with primers from exons 12 to 15, failed to show any aberrantly spliced transcripts.

### Example 7

### Functional Consequences of the S662G Mutation

Studies of the doubletime mutant in Drosophila and the tau mutant in the golden hamster indicate that mutations affecting the function of casein kinase Iε significantly disrupt endogenous circadian clock function leading to altered period lengths or arrhythmicity. Kloss, B., et al., The Drosophila clock gene double-time encodes a protein closely related to human case in kinase l epsilon, Cell, 1998, 94(1):83-95; Lowrey, P. L., et al., Positional syntenic cloning and functional characterization of the mammalian circadian mutation tau, Science, 2000, 288(5465):483-92. In addition, hPER2 and mPER2 are substrates of casein kinase Iε. Lowrey, P. L., et al., Positional syntenic cloning and functional characterization of the mammalian circadian mutation tau, Science, 2000, 288(5465):483-92, (and D.V., unpublished data). The CKIε binding site on hPER2 was mapped (FIG. 4) and it was determined that the S662G mutation fell within the CKIε binding region (FIG. 5). hPER2 and mPER2 fragments extending from amino acids 474 to 815 and 472 to 804, which encompass the CKIε binding region were used. It was reasoned that this mutant (662 S→G), which is located in the fragment phosphorylated by casein kinase Iε, eliminates a potential phosphorylation site and could alter any observed mobility shift. The amino acid sequence of the mutant hPER2 polypeptide is included herein in SEQ ID NO: 1. The nucleic acid sequence of this gene is included in SEQ ID NO: 2. In addition, the wild type hPER2 polypeptide sequence instead has a serine at position 662, and is found in SEQ ID NO: 3. The nucleic acid sequence of the wild type gene is found in SEQ ID NO: 4.

When reticulocyte lysates containing 35S-labeled hPER2 fragments were incubated with a low concentration of CKIε (0.25 ng/μl), an electrophoretic mobility shift was observed with the wild-type (662S) but not with the mutant (662G) fragment (FIG. 7A). A similar result was obtained with wild-type and mutant mPER2 fragments (data not shown). Phosphatase treatment confirmed that this shift was due to phosphorylation (FIG. 7A). When the experiment was repeated with a higher concentration of CKIε (6.5 ng/μl), mobility shifts were observed for both the wild type and mutant hPER2 fragments (FIG. 7B). No difference in the final extent of the shifts was observed due to limitations in the resolution obtainable on gel electrophoresis.

Analysis of the hPER2 sequence (FIG. 5) reveals several additional serine and threonine residues in the vicinity of 662S, which may be substrates for CKIε when the enzyme is present in excess. There is a precedent for the SXXS motif being a recognition site for CK1. Flotow, H., et al., Phosphate groups as substrate determinants for case in kinase I action, J. Biol. Chem., 1990, 265(24): 14264-9; Cegielska, A., et al., T-antigen kinase inhibits simian virus 40 DNA replication by phosphorylation of intact T-antigen on serines 120 and 123, J. Virol., 1994, 68(1):269-75; Sakaguchi, K., et al., Damage-mediated phosphorylation of human p53 threonine 18 through a cascade mediated by a case in 1-like kinase, Effect on Mdm2 binding, J. Biol. Chem., 2000, 275(13):9278-83; Dumaz, N., D. M. Milne, and D. W. Meek, Protein kinase CK1 is a p53-threonine 18 kinase which requires prior phosphorylation of serine 15, FEBS Lett., 1999, 463(3):312-6. The serine at position 662 does appear to be necessary for rapid hPER2 phosphorylation by CKIε. Phosphorylation of 662S could initiate a series of subsequent phosphorylations leading to the mobility shift observed when excess CKIε was applied.

To test this idea, the serine residue at position 662 was mutagenized to aspartate and glutamate, reasoning that the presence of a negative charge from the acidic residues would mimic a phosphoserine at position 662. Phosphorylation was restored in both the S662D and S662E mutants, with a higher phosphorylation rate observed with the S662D mutant (FIG. 7C). This was expected as the side chain of aspartate resembles that of phosphoserine more closely and would be a better “fit” with the presumed active site of CKIε. These results support the argument that phosphorylation of 662S regulates the phosphorylation of nearby residues. The phosphorylation sites and mechanisms for the spread of phosphorylation from 662S are currently being elucidated.

### Example 8

### Genetic Heterogeneity in FASPS

One other possible hPer2 mutation was identified in one small pedigree (intron base pair change, see above). Since no hPer2 mutations were identified in any of the four pedigrees shown in FIG. 3, genetic linkage analysis was performed in these families with polymorphic repeat markers within and closely flanking the hPer2 gene. Recombinants were identified in all four families, thus demonstrating that the ASPS in these families must be caused by mutations in other genes. Thus, like other model systems in which circadian rhythm mutants have been characterized, similar phenotypes can arise from mutations in different circadian rhythm genes. These families, along with additional families currently being collected, will prove an important resource for identifying other FASPS genes and mutations.

All references, publications, patents, patent applications, and commercial materials cited in this application are hereby incorporated by reference in their entirety. The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A method of diagnosing Familial Advanced Sleep Phase Syndrome in a human subject comprising:
screening for an alteration in a germline copy of the hPer2 gene of the human subject, wherein the detection of an alteration in the germline copy of the hPer2 gene of the human subject indicates a positive diagnosis of Familial Advanced Sleep Phase Syndrome in the human subject.

2. The method of claim 1, wherein the step of screening for an alteration in a germline copy of the hPer2 gene of the human subject comprises comparing the sequence of the germline copy of the hPer2 gene from the subject with a germline sequence of a wild-type hPer2 gene.

3. The method of claim 1, wherein the step of screening for an alteration in a germline copy of the hPer2 gene of the human subject comprises sequencing a germline copy of the hPer2 gene from the human subject and comparing the sequence of the germline copy of the hPer2 gene to a sequence with a known alteration in the hPer2 gene, wherein the sequence of the hPer2 gene that is substantially identical to the sequence with a known alteration in the hPer2 gene indicates an alteration in the germline copy of the hPer2 gene from the human subject.

4. The method of claim 3, wherein the sequence with a known alteration in the hPer2 gene is SEQ ID NO. 2.

5. The method of claim 2, further comprising the initial steps of obtaining a tissue sample from the human subject, isolating a germline copy of the hPer2 gene of the human subject from the tissue sample, and sequencing the isolated germline copy of the hPer2 gene prior to comparing the sequence of the germline copy of the hPer2 gene from the subject with a germline sequence of a wild-type hPer2 gene.

6. A method of diagnosing Familial Advanced Sleep Phase Syndrome in a human subject comprising:
screening for an alteration in the hPer2 polypeptide of the human subject, wherein the detection of an alteration in hPer2 polypeptide of the human subject indicates a positive diagnosis of Familial Advanced Sleep Phase Syndrome in the human subject.

7. The method of claim 6, wherein the step of screening for an alteration in the hPer2 polypeptide of the human subject comprises comparing the sequence of a hPer2 polypeptide from the subject with a wild-type hPer2 polypeptide.

8. The method of claim 6, wherein the step of screening for an alteration in the hPer2 polypeptide of the human subject comprises sequencing a hPer2 polypeptide from the human subject and comparing the sequence of the hPer2 polypeptide to a sequence with a known alteration in the hPer2 polypeptide, wherein the sequence of the hPer2 polypeptide that is identical to the sequence with a known alteration in the hPer2 polypeptide indicates an alteration in the hPer2 polypeptide from the human subject.

9. The method of claim 8, wherein the sequence with a known alteration in the hPer2 gene is SEQ ID NO. 3.

10. The method of claim 6, further comprising the initial steps of obtaining a tissue sample from the human subject, isolating a hPer2 polypeptide of the human subject from the tissue sample, and sequencing the isolated hPer2 polypeptide prior to comparing the sequence of the hPer2 polypeptide from the subject with a wild-type hPer2 polypeptide.

11. A method of diagnosing Familial Advanced Sleep Phase Syndrome in a human subject comprising:
screening for hypophosphorylation of hPer2 polypeptides of the human subject, wherein the detection of hypophosphorylation of hPer2 polypeptides of the human subject indicates a positive diagnosis of Familial Advanced Sleep Phase Syndrome in the human subject.

12. The method of claim 11, wherein the step of screening for hypophosphorylation of hPer2 polypeptides of the human subject comprises comparing the amount of phosphorylation of hPer2 polypeptides from the subject with the amount of phosphorylation of wild-type hPer2 polypeptides.

13. The method of claim 11, further comprising the initial step of obtaining hPer2 polypeptides from fibroblast cells taken from the human subject.

14. A method of screening for inhibitors of casein kinase epsilon comprising the steps of:
contacting a potential inhibitor of casein kinase I epsilon with casein kinase I epsilon in the presence of hPER2 and phosphates;
measuring the level of phosphorylation of the hPER2, wherein a level of phosphorylation observed with the potential inhibitor lower than a level of phosphorylation observed when casein kinase I epsilon is contacted with hPER2 and phosphates without the potential inhibitor signals an inhibitor of casein kinase I epsilon.

15. The method of claim 14, wherein the step of measuring the level of phosphorylation of the hPER2 comprises electrophoresis, wherein an electrophoretic mobility shift of the hPER2 resulting from the method from that observed with hPER2 not subjected to the method denotes phosphorylation.

16. The method of claim 15, further comprising the step of treating the hPER2 with phosphatase to confirm that the mobility shift is due to phosphorylation.

17. A method of screening for compounds which upregulate the phosphorylation of hPER2 by casein kinase I epsilon comprising the steps of:
contacting a potential upregulating compound with casein kinase I epsilon in the presence of hPER2 and phosphates;
measuring the level of phosphorylation of the hPER2, wherein a level of phosphorylation observed with the potential upregulating compound higher than a level of phosphorylation observed when casein kinase I epsilon is contacted with hPER2 and phosphates without the potential upregulating compound signals an upregulating compound for casein kinase I epsilon.

18. The method of claim 17, wherein the step of measuring the level of phosphorylation of the hPER2 comprises electrophoresis, wherein an electrophoretic mobility shift of the hPER2 resulting from the method from that observed with hPER2 not subjected to the method denotes phosphorylation.

19. The method of claim 17, further comprising the step of treating the hPER2 with phosphatase to confirm that the mobility shift is due to phosphorylation.

20. An isolated and purified nucleic acid molecule comprising a nucleotide sequence which encodes an amino acid sequence at least 80% identical to the amino acid sequence of SEQ ID NO: 1.

21. An isolated and purified nucleic acid molecule comprising a nucleotide sequence which encodes an amino acid sequence at least 90% identical to the amino acid sequence of SEQ ID NO: 1.

22. An isolated and purified nucleic acid molecule comprising a nucleotide sequence which encodes an amino acid sequence at least 95% identical to the amino acid sequence of SEQ ID NO: 1.

23. An isolated and purified nucleic acid molecule comprising a nucleotide sequence which encodes the amino acid sequence of SEQ ID NO: 1.

24. An isolated nucleic acid molecule having the sequence found in SEQ. ID NO: 2.

25. The nucleic acid molecule of claim 24, wherein the nucleic acid molecule is subcloned into a plasmid.

26. The nucleic acid molecule of claim 24, wherein the nucleic acid molecule is subcloned into a prokaryotic or eukaryotic expression vector.

27. The nucleic acid molecule of claim 24, wherein the nucleic acid molecule is operably linked to a heterologous promoter.

28. The nucleic acid molecule of claim 24, wherein the nucleic acid molecule is stably or transiently incorporated into a prokaryotic or eukaryotic host cell.

29. A method for treating advanced sleep phase syndrome of aging in a human subject comprising administering AzaC to the human subject.

30. A method for treating advanced sleep phase syndrome of aging in a human subject comprising administering a histone deacetylase inhibitor to the subject.

31. A method of screening for inhibitors of casein kinase I delta comprising the steps of:
contacting a potential inhibitor of casein kinase I delta with casein kinase I delta in the presence of hPER2 and phosphates;
measuring the level of phosphorylation of the hPER2, wherein a level of phosphorylation observed with the potential inhibitor lower than a level of phosphorylation observed when casein kinase delta is contacted with hPER2 and phosphates without the potential inhibitor signals an inhibitor of casein kinase I delta.

32. The method of claim 31, wherein the step of measuring the level of phosphorylation of the hPER2 comprises electrophoresis, wherein an electrophoretic mobility shift of the hPER2 resulting from the method from that observed with hPER2 not subjected to the method denotes phosphorylation.

33. The method of claim 32, further comprising the step of treating the hPER2 with phosphatase to confirm that the mobility shift is due to phosphorylation.

34. A method of screening for compounds which upregulate the phosphorylation of hPER2 by casein kinase I delta comprising the steps of:
contacting a potential upregulating compound with casein kinase I delta in the presence of hPER2 and phosphates;
measuring the level of phosphorylation of the hPER2, wherein a level of phosphorylation observed with the potential upregulating compound higher than a level of phosphorylation observed when casein kinase I delta is contacted with hPER2 and phosphates without the potential upregulating compound signals an upregulating compound for casein kinase I delta.

35. The method of claim 34, wherein the step of measuring the level of phosphorylation of the hPER2 comprises electrophoresis, wherein an electrophoretic mobility shift of the hPER2 resulting from the method from that observed with hPER2 not subjected to the method denotes phosphorylation.

36. The method of claim 35, further comprising the step of treating the hPER2 with phosphatase to confirm that the mobility shift is due to phosphorylation.

37. A method of screening for compounds that inhibit the phosphorylation of hPER2 comprising the steps of:
contacting a potential inhibitor of the phosphorylation of hPER2 with hPER2 in the presence of a kinase and phosphates;
measuring the level of phosphorylation of the hPER2, wherein a level of phosphorylation observed with the potential inhibitor of the phosphorylation of hPER2 lower than a level of phosphorylation observed when hPER2 is contacted with a kinase and phosphates without the potential inhibitor of the phosphorylation of hPER2 signals an inhibitor of the phosphorylation of hPER2.

38. The method of claim 37, wherein the kinase is casein kinase I epsilon.

39. The method of claim 37, wherein the kinase is casein kinase delta.

40. The method of claim 37, wherein the step of measuring the level of phosphorylation of the hPER2 comprises electrophoresis, wherein an electrophoretic mobility shift of the hPER2 resulting from the method from that observed with hPER2 not subjected to the method denotes phosphorylation.

41. The method of claim 40, further comprising the step of treating the hPER2 with phosphatase to confirm that the mobility shift is due to phosphorylation.

42. A method of screening for compounds that upregulate the phosphorylation of hPER2 comprising the steps of:
contacting a potential upregulating compound of the phosphorylation of hPER2 with hPER2 in the presence of a kinase and phosphates;
measuring the level of phosphorylation of the hPER2, wherein a level of phosphorylation observed with the potential upregulating compound of the phosphorylation of hPER2 higher than a level of phosphorylation observed when hPER2 is contacted with a kinase and phosphates without the potential upregulating compound of the phosphorylation of hPER2 signals an upregulating compound for the phosphorylation of hPER2.

43. The method of claim 37, wherein the kinase is casein kinase I epsilon.

44. The method of claim 37, wherein the kinase is casein kinase delta.

45. The method of claim 42, wherein the step of measuring the level of phosphorylation of the hPER2 comprises electrophoresis, wherein an electrophoretic mobility shift of the hPER2 resulting from the method from that observed with hPER2 not subjected to the method denotes phosphorylation.

46. The method of claim 45, further comprising the step of treating the hPER2 with phosphatase to confirm that the mobility shift is due to phosphorylation.


SEQUENCE LISTING (10 sequences)

SEQ ID NO: 1 - protein, 1255 aa, HOMO SAPIENS
MNGYAEFPPSPSNPTKEPVEPQPSQVPLQEDVDMSSGSSGHETNENCSTGRDSQGSDCDDSGKELGMLVEPPDARQSPDTFSLMMAKSEHNPSTSGCSSDQSSKVDTHKELIKTLKELKVHLPADKKAKGKASTLATLKYALRSVKQVKANEEYYQLLMSSEGHPCGADVPSYTVEEMESVTSEHIVKNADMFAVAVSLVSGKILYISDQVASIFHCKRDAFSDAKFVEFLAPHDVGVFHSFTSPYKLPLWSMCSGADSFTQECMEEKSFFCRVSVRKSHENEIRYHPFRMTPYLVKVRDQQGAESQLCCLLLAERVHSGYEAPRIPPEKRIFTTTHTPNCLFQDVDERAVPLLGYLPQDLIETPVLVQLHPSDRPLMLAIHKKILQSGGQPFDYSPIRFRARNGEYITLDTSWSSFINPWSRKISFIIGRHKVRVGPLNEDVFAAHPCTEEKALHPSIQELTEQIHRLLLQPVPHSGSSGYGSLGSNGSHEHLMSQTSSSDSNGHEDSRRRRAEICKNGNKTKNRSHYSHESGEQKKKSVTEMQTNPPAEKKAVPAMEKDSLGVSFPEELACKNQPTCSYQQISCLDSVIRYLESCNEAATLKRKCEFPANVPALRSSDKRKATVSPGPHAGEAEPPSRVNSRTGVGTHLTSLALPGKAEGVASLTSQCSYSSTIVHVGDKKPQPELEMVEDAASGPESLDCLAGPALACGLSQEKEPFKKLGLTKEVLAAHTQKEEQSFLQKFKEIRKLSIFQSHCHYYLQERSKGQPSERTAPGLRNTSGIDSPWKKTGKNRKLKSKRVKPRDSSESTGSGGPVSARPPLVGLNATAWSPSDTSQSSCPAVPFPAPVPAAYSLPVFPAPGTVAAPPAPPHASFTVPAVPVDLQHQFAVQPPPFPAPLAPVMAFMLPSYSFPSGTPNLPQAFFPSQPQFPSHPTLTSEMASASQPEFPSRTSIPRQPCACPATRATPPSAMGRASPPLFQSRSSSPLQLNLLQLEEAPEGGTGAMGTTGATETAAVGADCKPGTSRDQQPKAPLTRDEPSDTQNSDALSTSSGLLNLLLNEDLCSASGSAASESLGSGSLGCDASPSGAGSSDTSHTSKYFGSIDSSENNHKAKMNTGMEESEHFIKCVLQDPIWLLMADADSSVMMTYQLPSRNLEAVLKEDREKLKLLQKLQPRFTESQKQELREVHQWMQTGGLPAAIDVAECVYCENKEKGNICIPYEEDIPSLGLSEVSDTKEDENGSPLNHRIEEQT

SEQ ID NO: 2 - DNA, 6216 bp, HOMO SAPIENS
gtcgagccgcgcgcagggtgcgctcgtttgaactgcggtgacaccgagggttggggactcgaacccccgcttcgcagctcaggagcctgaggtccgaaagcttcgttccagagcccagcatgaatggatacgcggaatttccgcccagccccagtaaccccaccaaggagcccgtggagccccagcccagccaggtcccactgcaggaagatgtggacatgagcagtggctccagtggacatgagaccaacgaaaactgctccacggggcgggactcgcagggcagtgactgtgacgacagtgggaaggagctggggatgctggtggagccaccggatgcccgccagagtccagatacctttagcctgatgatggcaaaatctgaacacaacccatctacaagtggctgcagtagcgaccagtcttcgaaagtggacacacacaaagaactgataaaaacactaaaggagctgaaggtccacctccctgcagacaagaaggccaagggcaaggccagtacgctggccaccttgaagtacgccctcaggagcgtgaagcaggtgaaagccaatgaagagtattaccagctgctgatgtccagcgagggtcacccctgtggagcagacgtgccctcctacaccgtggaggagatggagagcgttacctctgagcacattgtgaagaatgccgatatgtttgcggtggccgtgtccctggtgtctgggaagatcctgtacatctctgaccaggttgcatccatatttcactgtaaaagagatgccttcagcgatgccaagtttgtggagttcctggcgcctcacgatgtgggcgtgttccacagtttcacctccccgtacaagcttcccttgtggagcatgtgcagtggagcagattcttttactcaagaatgcatggaggagaaatctttcttttgccgtgtcagtgtccggaaaagccacgagaatgaaatccgctaccaccccttccgcatgacgccctacctggtcaaggtgcgggaccaacaaggtgctgagagtcagctttgctgccttctgctggcagagagagtgcactctggttatgaagcccctagaattcctcctgaaaagagaatttttacaaccacccatacaccaaattgtttgttccaggatgtggatgaaagggcggtccctctcctgggctacctacctcaggacctgattgaaaccccagtgctcgtgcagctccaccctagtgacaggcccttgatgctggccatccacaaaaagatcctgcagtcaggcgggcagcctttcgactattctcccattcggtttcgcgcccggaacggagagtacatcacgttggacaccagctggtccagcttcatcaacccatggagcaggaaaatctccttcatcattgggaggcacaaagtcagggtgggccctttgaatgaggacgtgtttgcagcccacccctgcacagaggagaaggccctgcaccccagcattcaggagctcacagagcagatccaccggctcctgctgcagcccgtcccccacagcggctccagtggctacgggagtctgggcagcaacgggtcccacgagcaccttatgagccagacctcctccagcgacagcaacggccatgaggactcacgccggaggagagccgaaatttgtaaaaatggtaacaagaccaaaaatagaagtcattattctcatgaatctggagaacaaaagaaaaaatccgttacagaaatgcaaactaatcccccagctgagaagaaagctgtccctgccatggaaaaggacagcctgggggtcagcttccccgaggagttggcctgcaagaaccagcccacctgctcctaccagcagatcagctgcttggacagcgtcatcaggtacttggagagctgcaatgaggctgccaccctgaagaggaaatgcgagttcccagcaaacgtcccagcgctaaggtccagtgataagcggaaggccacagtcagcccagggccacacgctggagaggcagagccgccctccagggtgaacagccgcacgggagtaggtacgcacctgacctcgctggcactgccgggcaaggcagagggtgtggcgtcgctcaccagccagtgcagctacagcagcaccatcgtccatgtgggagacaagaagccgcagccggagttagagatggtggaagatgctgcgagtgggccagaatccctggactgcctggcgggccctgccctggcctgtggtctcagccaagagaaggagcccttcaagaagctgggcctcaccaaggaggtactcgctgcacacacacagaaggaggagcagagcttcctgcagaagttcaaagaaataagaaaactcagcattttccagtcccactgccattactacttgcaagaaagatccaaggggcagccaagtgaacgaactgcccctggactaagaaatacttccggaatagattcaccttggaaaaaaacaggaaagaacagaaaattgaagtccaagcgggtcaaacctcgagactcatctgagagcaccggatctggggggcccgtgtccgcccggcccccgctggtgggcttgaacgccacagcctggtcaccctcagacacgtcccagtccagctgcccagccgtgccctttcccgccccagtgccagcagcttattcactgcccgtgtttccagcgccagggactgtggcagcacccccggcacctccccacgccagcttcacagtgcctgctgtgcccgtggacctccagcaccagtttgcagtccagcccccacctttccctgcccctttggcgcctgtcatggcattcatgctacccagttattccttcccctcggggaccccaaacctgccccaggccttcttccccagccagcctcagtttccgagccaccccacactcacatccgagatggcctctgcctcacagcctgagttccccagccggacctcgatccccagacagccatgtgcttgtccagccacccgggccaccccaccatcggccatgggtagggcctccccaccgctctttcagtcccgcagcagctcgcccctgcagctcaacctgctgcagctggaggaagcccctgagggtggcactggagccatggggaccacaggggccacagagacagcagctgtaggggcggactgcaaacctggcacttctcgggaccagcagccgaaggcgcctctgacccgtgatgaaccctcagacacacagaacagtgacgccctttccacgtcaagcggcctcctaaacctcctgctgaatgaggacctctgctcagcctcgggctctgctgcttcggagtctctgggctccggctcactgggctgcgacgcctccccgagtggggcaggcagtagtgacacaagtcataccagcaaatattttggaagcattgactcctcagagaataatcacaaagcaaaaatgaacactggtatggaagaaagtgagcatttcattaagtgcgtcctgcaggatcccatctggctgctgatggcagatgcggacagcagcgtcatgatgacgtaccagctgccttcccgaaatttagaagcggttttgaaggaggacagagagaagctgaagctcctacagaaactccagcccaggttcacggagagtcagaagcaggagctgcgcgaggtccaccagtggatgcagacgggcggcctgcccgcagccatcgacgtggcagaatgtgtttactgtgaaaacaaggaaaaaggtaatatttgcataccatatgaggaagatattccttctctgggactcagcgaagtgtcggacaccaaagaagacgaaaatggatcccccttgaatcacaggatcgaagagcagacgtaacccctgccccacctcagcccggcagccagcgaggtacaccaggtggtgcttggaagagatgaaagatcttcatggctgtttccactgaaatggacacatatgctcatgttgctttttttgttttagaaaaaaaaacaacatagttttctgaaggggcgacttaaaactgtggagagtggggagagttcggaaagaaatatgtttttatatataaaatatatatgtggagttttgtgggatggggaagagattttagttgttatttaacttgagaaagactaagcgcctcttagtgtcagggaagttgcctcagtgctcccagaagtcctgtgactgtgacgagacctctgtctgctgcaccagctggggactctggcttccagagctttcccagggtgtttggatcagatcaaattttgtcctctcttggggactgctttttatctgaattatcatttagtcaaggtagagtgtttttttatacataccaaatggagatagcagcctctcctagttttatttcaaaacgtttcacattaaatggtgtgaagcgttgtttggcaaaccaacagctttggcttctggtgtggtcaatatttcagtctgacataggttttgtttgtagtgaacaaagttgaaacatttgctctggactaaagaagcctagtggtttgtgtggccaactccatcggatgaatgcacacgcagacagaccctctgtatatttctgcattattcttgtctccttttcagaccatgatggccaatatggagattaaaatatgtcatcagtcatctctttatggtgacttccctttgcaaaccaggctgtgaccaacacatgtgagacccagtcctgtttggttttcttccgttggaaccacccagacatctgcttccacccagccaagcccacatcacatctcctggccgagagcagccactgccactcagtctgacagcttgcgactgcatctgtattttcaggggtgcagtgagctcacctctcccactgcaccctgggttgggtgcacagccctcattcttttcatgagcccgacctctctcggagcagcttcaggcctctgccagtgtccccagcacttttaggtcatttggacacttggggaaaagtgaggccagtctgcccggctttttacaaaacctcatgttgcattgtatattccaaagatggttcagaaaatttaatattggtccctggtggaaattcaaagttatcactgaagaacagttgacttaaaattggaccaagactatgaggcttaaaagggaccagggttttctttttttttttttttttttttttttttagatggagtttctttttgcccaggctggagtgcagtggcgccatcttggctcactgcaacctctgcctcccaggttcaagcgattctcctgcctcagcctcctgagtagctgggaccacaggcgactgccaccacacccagctaattttttgtatttttagtagagacagggtttcaccatgttggccaggctggtctcgaactcctgacctcaagcgatccacccacctcggcctcccaaagtgctgggattacaggcgtgagccaccacgcccaactgggaccagggttttctgttttttgatggaggtgaaatctctttgtaatccactaggttttcatcgtaaaaccatcttatgcctgactattaaacctattcttcataaacacaagaacactttaatttttcgttaatttacaaagtaacatcagctgcctatgcctatgataaggtagcagtctgcattcttatggccattagatgttacaaactccttgcctctaaagtcagatcatgaagggataggtgttcatctaaggttacagttatgttaccgaaacacaaaactgccaaaatcttactctgctgttatgaatgtttaccatcagcattattttatcatttaatatgtgctcactgattgttaactgtagcttcagcgcgtgccaagcagttgacttaataggatcatcttgtgaatttgtttacgtgatgccaagcatcaagtcatgttttctttagtgtgtgtgcttacacaggtgttaaacagtttttctctattttaaactgagccttctttttaatatattcccgaagagatatgtaaataagctctcagagtttctgtgatgatttgttgagccttgctggacaagtggtttgtttgtgtgcaaaccaaactttctttacccagtgcaatagatttgtttgactgcttgtgtctttttatgacctgtttgccttttagaaaattggtaaataaagcaagtatattttt

SEQ ID NO: 3 - protein, 1255 aa, HOMO SAPIENS
MNGYAEFPPSPSNPTKEPVEPQPSQVPLQEDVDMSSGSSGHETNENCSTGRDSQGSDCDDSGKELGMLVEPPDARQSPDTFSLMMAKSEHNPSTSGCSSDQSSKVDTHKELIKTLKELKVHLPADKKAKGKASTLATLKYALRSVKQVKANEEYYQLLMSSEGHPCGADVPSYTVEEMESVTSEHIVKNADMFAVAVSLVSGKILYISDQVASIFHCKRDAFSDAKFVEFLAPHDVGVFHSFTSPYKLPLWSMCSGADSFTQECMEEKSFFCRVSVRKSHENEIRYHPFRMTPYLVKVRDQQGAESQLCCLLLAERVHSGYEAPRIPPEKRIFTTTHTPNCLFQDVDERAVPLLGYLPQDLIETPVLVQLHPSDRPLMLAIHKKILQSGGQPFDYSPIRFRARNGEYITLDTSWSSFINPWSRKISFIIGRHKVRVGPLNEDVFAAHPCTEEKALHPSIQELTEQIHRLLLQPVPHSGSSGYGSLGSNGSHEHLMSQTSSSDSNGHEDSRRRRAEICKNGNKTKNRSHYSHESGEQKKKSVTEMQTNPPAEKKAVPAMEKDSLGVSFPEELACKNQPTCSYQQISCLDSVIRYLESCNEAATLKRKCEFPANVPALRSSDKRKATVSPGPHAGEAEPPSRVNSRTGVGTHLTSLALPGKAESVASLTSQCSYSSTIVHVGDKKPQPELEMVEDAASGPESLDCLAGPALACGLSQEKEPFKKLGLTKEVLAAHTQKEEQSFLQKFKEIRKLSIFQSHCHYYLQERSKGQPSERTAPGLRNTSGIDSPWKKTGKNRKLKSKRVKPRDSSESTGSGGPVSARPPLVGLNATAWSPSDTSQSSCPAVPFPAPVPAAYSLPVFPAPGTVAAPPAPPHASFTVPAVPVDLQHQFAVQPPPFPAPLAPVMAFMLPSYSFPSGTPNLPQAFFPSQPQFPSHPTLTSEMASASQPEFPSRTSIPRQPCACPATRATPPSAMGRASPPLFQSRSSSPLQLNLLQLEEAPEGGTGAMGTTGATETAAVGADCKPGTSRDQQPKAPLTRDEPSDTQNSDALSTSSGLLNLLLNEDLCSASGSAASESLGSGSLGCDASPSGAGSSDTSHTSKYFGSIDSSENNHKAKMNTGMEESEHFIKCVLQDPIWLLMADADSSVMMTYQLPSRNLEAVLKEDREKLKLLQKLQPRFTESQKQELREVHQWMQTGGLPAAIDVAECVYCENKEKGNICIPYEEDIPSLGLSEVSDTKEDENGSPLNHRIEEQT

SEQ ID NO: 4 - DNA, 6219 bp, HOMO SAPIENS
gtggtcgagccgcgcgcagggtgcgctcgtttgaactgcggtgacaccgagggttggggactcgaacccccgcttcgcagctcaggagcctgaggtccgaaagcttcgttccagagcccagcatgaatggatacgcggaatttccgcccagccccagtaaccccaccaaggagcccgtggagccccagcccagccaggtcccactgcaggaagatgtggacatgagcagtggctccagtggacatgagaccaacgaaaactgctccacggggcgggactcgcagggcagtgactgtgacgacagtgggaaggagctggggatgctggtggagccaccggatgcccgccagagtccagatacctttagcctgatgatggcaaaatctgaacacaacccatctacaagtggctgcagtagcgaccagtcttcgaaagtggacacacacaaagaactgataaaaacactaaaggagctgaaggtccacctccctgcagacaagaaggccaagggcaaggccagtacgctggccaccttgaagtacgccctcaggagcgtgaagcaggtgaaagccaatgaagagtattaccagctgctgatgtccagcgagggtcacccctgtggagcagacgtgccctcctacaccgtggaggagatggagagcgttacctctgagcacattgtgaagaatgccgatatgtttgcggtggccgtgtccctggtgtctgggaagatcctgtacatctctgaccaggttgcatccatatttcactgtaaaagagatgccttcagcgatgccaagtttgtggagttcctggcgcctcacgatgtgggcgtgttccacagtttcacctccccgtacaagcttcccttgtggagcatgtgcagtggagcagattcttttactcaagaatgcatggaggagaaatctttcttttgccgtgtcagtgtccggaaaagccacgagaatgaaatccgctaccaccccttccgcatgacgccctacctggtcaaggtgcgggaccaacaaggtgctgagagtcagctttgctgccttctgctggcagagagagtgcactctggttatgaagcccctagaattcctcctgaaaagagaatttttacaaccacccatacaccaaattgtttgttccaggatgtggatgaaagggcggtccctctcctgggctacctacctcaggacctgattgaaaccccagtgctcgtgcagctccaccctagtgacaggcccttgatgctggccatccacaaaaagatcctgcagtcaggcgggcagcctttcgactattctcccattcggtttcgcgcccggaacggagagtacatcacgttggacaccagctggtccagcttcatcaacccatggagcaggaaaatctccttcatcattgggaggcacaaagtcagggtgggccctttgaatgaggacgtgtttgcagcccacccctgcacagaggagaaggccctgcaccccagcattcaggagctcacagagcagatccaccggctcctgctgcagcccgtcccccacagcggctccagtggctacgggagtctgggcagcaacgggtcccacgagcaccttatgagccagacctcctccagcgacagcaacggccatgaggactcacgccggaggagagccgaaatttgtaaaaatggtaacaagaccaaaaatagaagtcattattctcatgaatctggagaacaaaagaaaaaatccgttacagaaatgcaaactaatcccccagctgagaagaaagctgtccctgccatggaaaaggacagcctgggggtcagcttccccgaggagttggcctgcaagaaccagcccacctgctcctaccagcagatcagctgcttggacagcgtcatcaggtacttggagagctgcaatgaggctgccaccctgaagaggaaatgcgagttcccagcaaacgtcccagcgctaaggtccagtgataagcggaaggccacagtcagcccagggccacacgctggagaggcagagccgccctccagggtgaacagccgcacgggagtaggtacgcacctgacctcgctggcactgccgggcaaggcagagagtgtggcgtcgctcaccagccagtgcagctacagcagcaccatcgtccatgtgggagacaagaagccgcagccggagttagagatggtggaagatgctgcgagtgggccagaatccctggactgcctggcgggccctgccctggcctgtggtctcagccaagagaaggagcccttcaagaagctgggcctcaccaaggaggtactcgctgcacacacacagaaggaggagcagagcttcctgcagaagttcaaagaaataagaaaactcagcattttccagtcccactgccattactacttgcaagaaagatccaaggggcagccaagtgaacgaactgcccctggactaagaaatacttccggaatagattcaccttggaaaaaaacaggaaagaacagaaaattgaagtccaagcgggtcaaacctcgagactcatctgagagcaccggatctggggggcccgtgtccgcccggcccccgctggtgggcttgaacgccacagcctggtcaccctcagacacgtcccagtccagctgcccagccgtgccctttcccgccccagtgccagcagcttattcactgcccgtgtttccagcgccagggactgtggcagcacccccggcacctccccacgccagcttcacagtgcctgctgtgcccgtggacctccagcaccagtttgcagtccagcccccacctttccctgcccctttggcgcctgtcatggcattcatgctacccagttattccttcccctcggggaccccaaacctgccccaggccttcttccccagccagcctcagtttccgagccaccccacactcacatccgagatggcctctgcctcacagcctgagttccccagccggacctcgatccccagacagccatgtgcttgtccagccacccgggccaccccaccatcggccatgggtagggcctccccaccgctctttcagtcccgcagcagctcgcccctgcagctcaacctgctgcagctggaggaagcccctgagggtggcactggagccatggggaccacaggggccacagagacagcagctgtaggggcggactgcaaacctggcacttctcgggaccagcagccgaaggcgcctctgacccgtgatgaaccctcagacacacagaacagtgacgccctttccacgtcaagcggcctcctaaacctcctgctgaatgaggacctctgctcagcctcgggctctgctgcttcggagtctctgggctccggctcactgggctgcgacgcctccccgagtggggcaggcagtagtgacacaagtcataccagcaaatattttggaagcattgactcctcagagaataatcacaaagcaaaaatgaacactggtatggaagaaagtgagcatttcattaagtgcgtcctgcaggatcccatctggctgctgatggcagatgcggacagcagcgtcatgatgacgtaccagctgccttcccgaaatttagaagcggttttgaaggaggacagagagaagctgaagctcctacagaaactccagcccaggttcacggagagtcagaagcaggagctgcgcgaggtccaccagtggatgcagacgggcggcctgcccgcagccatcgacgtggcagaatgtgtttactgtgaaaacaaggaaaaaggtaatatttgcataccatatgaggaagatattccttctctgggactcagcgaagtgtcggacaccaaagaagacgaaaatggatcccccttgaatcacaggatcgaagagcagacgtaacccctgccccacctcagcccggcagccagcgaggtacaccaggtggtgcttggaagagatgaaagatcttcatggctgtttccactgaaatggacacatatgctcatgttgctttttttgttttagaaaaaaaaacaacatagttttctgaaggggcgacttaaaactgtggagagtggggagagttcggaaagaaatatgtttttatatataaaatatatatgtggagttttgtgggatggggaagagattttagttgttatttaacttgagaaagactaagcgcctcttagtgtcagggaagttgcctcagtgctcccagaagtcctgtgactgtgacgagacctctgtctgctgcaccagctggggactctggcttccagagctttcccagggtgtttggatcagatcaaattttgtcctctcttggggactgctttttatctgaattatcatttagtcaaggtagagtgtttttttatacataccaaatggagatagcagcctctcctagttttatttcaaaacgtttcacattaaatggtgtgaagcgttgtttggcaaaccaacagctttggcttctggtgtggtcaatatttcagtctgacataggttttgtttgtagtgaacaaagttgaaacatttgctctggactaaagaagcctagtggtttgtgtggccaactccatcggatgaatgcacacgcagacagaccctctgtatatttctgcattattcttgtctccttttcagaccatgatggccaatatggagattaaaatatgtcatcagtcatctctttatggtgacttccctttgcaaaccaggctgtgaccaacacatgtgagacccagtcctgtttggttttcttccgttggaaccacccagacatctgcttccacccagccaagcccacatcacatctcctggccgagagcagccactgccactcagtctgacagcttgcgactgcatctgtattttcaggggtgcagtgagctcacctctcccactgcaccctgggttgggtgcacagccctcattcttttcatgagcccgacctctctcggagcagcttcaggcctctgccagtgtccccagcacttttaggtcatttggacacttggggaaaagtgaggccagtctgcccggctttttacaaaacctcatgttgcattgtatattccaaagatggttcagaaaatttaatattggtccctggtggaaattcaaagttatcactgaagaacagttgacttaaaattggaccaagactatgaggcttaaaagggaccagggttttctttttttttttttttttttttttttttagatggagtttctttttgcccaggctggagtgcagtggcgccatcttggctcactgcaacctctgcctcccaggttcaagcgattctcctgcctcagcctcctgagtagctgggaccacaggcgactgccaccacacccagctaattttttgtatttttagtagagacagggtttcaccatgttggccaggctggtctcgaactcctgacctcaagcgatccacccacctcggcctcccaaagtgctgggattacaggcgtgagccaccacgcccaactgggaccagggttttctgttttttgatggaggtgaaatctctttgtaatccactaggttttcatcgtaaaaccatcttatgcctgactattaaacctattcttcataaacacaagaacactttaatttttcgttaatttacaaagtaacatcagctgcctatgcctatgataaggtagcagtctgcattcttatggccattagatgttacaaactccttgcctctaaagtcagatcatgaagggataggtgttcatctaaggttacagttatgttaccgaaacacaaaactgccaaaatcttactctgctgttatgaatgtttaccatcagcattattttatcatttaatatgtgctcactgattgttaactgtagcttcagcgcgtgccaagcagttgacttaataggatcatcttgtgaatttgtttacgtgatgccaagcatcaagtcatgttttctttagtgtgtgtgcttacacaggtgttaaacagtttttctctattttaaactgagccttctttttaatatattcccgaagagatatgtaaataagctctcagagtttctgtgatgatttgttgagccttgctggacaagtggtttgtttgtgtgcaaaccaaactttctttacccagtgcaatagatttgtttgactgcttgtgtctttttatgacctgtttgccttttagaaaattggtaaataaagcaagtatattttt

SEQ ID NO: 5 - protein, 41 aa, HOMO SAPIENS
EPVVGGTLSPLALANKAESVVSVTSQCSFSSTIVHVGDKKP

SEQ ID NO: 6 - protein, 41 aa, MUS MUSCULUS; identical to SEQ ID NO: 5

SEQ ID NO: 7 - protein, 41 aa, HOMO SAPIENS
RTGVGTHLTSLALPGKAESVASLTSQCSYSSTIVHVGDKKP

SEQ ID NO: 8 - protein, 41 aa, MUS MUSCULUS
HTEVSAHLSSLTLPGKAESVVSLTSQCSYSSTIVHVGDKKP

SEQ ID NO: 9 - protein, 41 aa, HOMO SAPIENS
RSIDTGGGAPQILSTAMLSLGSGISQCGYSSTIVHVPPPET

SEQ ID NO: 10 - protein, 41 aa, MUS MUSCULUS
PSTDIEGGAARTLSTAALSVASGISQCSCSSTSGHAPPLQS